(19) United States
(12) Patent Application Publication
(10) Pub. No.: US 2023/0243001 A1
(43) Pub. Date: Aug. 3, 2023

(54) Detection of Macrolide-Resistant Mycoplasma Genitalium

(71) Applicant: Gen-Probe Incorporated, San Diego, CA (US)

(72) Inventors: Barbara L. Eaton, San Diego, CA (US); Benjamin Grobarczyk, Saive (BE); Laurent Franzil, Flémalle (BE)

(73) Assignee: Gen-Probe Incorporated, San Diego, CA (US)

(21) Appl. No.: 18/015,619

(22) PCT Filed: Jul. 19, 2021
(86) PCT No.: PCT/US2021/042222

§ 371 Date: Jan. 11, 2023

Related U.S. Application Data
(60) Provisional application No. 63/053,232, filed on Jul. 17, 2020.

(51) Int. Cl. C12Q 1/689

(52) U.S. Cl. CPC C12Q 1/689; C12Q 2600/106; C12Q 2600/156

(57) ABSTRACT

Provided are methods, compositions, and systems for detecting nucleic acids of macrolide-resistant Mycoplasma geni-talium using FRET probes for detecting SNPs at position 2058 and 2059.

### CROSS-REFERENCE TO RELATED APPLICATION

This application claims the benefit under 35 U.S.C. §119(e) of U.S. Provisional Application No. 63/053,232, filed Jul. 17, 2020. The entire disclosure of this earlier application is hereby incorporated by reference.

### TECHNICAL FIELD

The disclosure relates generally to the field of biotechnology. More specifically, the disclosure relates to compositions, methods, kits, and systems for detecting macrolide-resistant Mycoplasma genitalium.

### BACKGROUND

Mycoplasmas are small prokaryotic organisms (0.2 to 0.3 µm) belonging to the class Mollicutes, whose members lack a cell wall and have a small genome size. The mollicutes include at least 100 species of Mycoplasma, 13 of which are known to infect humans.

One Mycoplasma species of clinical relevance is M. genitalium. This organism, which is thought to be a cause of sexually transmitted nongonococcal urethritis (NGU), has been detected to a significantly greater extent in symptomatic males than in asymptomatic males. See Yoshida et al., “Phylogeny-Based Rapid Identification of Mycoplasma and Ureaplasmas from Urethritis Patients,” J. Clin. Microbiol., 40:105-110 (2002). In addition to NGU, M. genitalium is thought to be involved in pelvic inflammatory disease (which can lead to infertility in women in severe cases), adverse birth outcomes, and increased risk for human immunodeficiency virus (HIV) infection. See Maniloff et al., Mycoplasmas: Molecular Biology and Pathogenesis 417 (ASM 1992); and Manhart et al., supplement to Contemporary OB/GYN (July 2017).

Significantly, M. genitialium is more common than many other sexually transmitted pathogens. Studies of low-risk individuals estimated the prevalence of M. genitialium among women to be in the range of from 0.8% - 4.1%, and among men to be in the range of from 1.1% - 1.2%. Among the population of women attending an STI clinic, the prevalence of M. genitialium ranged as high as 19% in two major U.S. cities. The prevalence was as high as 15% for men attending the STI clinics. In recent studies, M. genitialium prevalence was higher than all other bacterial sexually transmitted infections.

The advent and spread of antibiotic-resistant strains of M. genitalium greatly complicates infection control. Current treatment protocols for M. genitialium infection rely on administration of the macrolide antibiotic azithromycin. One study conducted in Australia more than a decade ago revealed evidence for progressive dissemination of M. genitialium bacteria that were resistant to this treatment. The resistance was attributed to adjacent mutations at two positions in the 23S rRNA that could be detected using nucleic acid sequencing or “high resolution melt analysis” techniques. Unfortunately, nucleic acid sequencing approaches do not lend themselves to rapid testing, and melt curve analyses, although effective, had trouble differentiating genotypes (i.e., wild-type and mutants). Benefits of early detection include the opportunity to reduce transmission of resistant M. genitialium strains in the community, and shortening the time to effective second line treatment. (See Twin et al., PLoS ONE 7(4): e35593. Doi:10.1371/joumal.pone.0035593)

Sensitive and specific molecular tests for nucleic acids of M. genitialium have been described in U.S. Pat. No. 7,345,155, the disclosure of which is incorporated by reference. However, these tests do not detect the macrolide resistance genetic marker. The present disclosure provides supplemental techniques that can be used for detecting the genetic marker of macrolide resistance in M. genitialium.

### SUMMARY OF THE DISCLOSURE

In a first aspect, the disclosure relates to a method of determining whether a nucleic acid sample isolated from a specimen obtained from a human subject includes nucleic acids of macrolide-resistant M. genitalium. Generally speaking, the method includes the steps of: (a) amplifying or having amplified 23S ribosomal nucleic acid sequences that may present in the nucleic acid sample using an in vitro nucleic acid amplification reaction to produce amplicons. The in vitro nucleic acid amplification reaction can include each of (i) a DNA polymerase with 5′ to 3′ exonuclease activity, (ii) a primer complementary to 23S ribosomal nucleic acids of both macrolide-resistant M. genitalium and macrolide-sensitive M. genitalium, and (iii) a collection of two or more oligonucleotide probes, where the base sequence of at least one oligonucleotide probe among the collection is selected from the group consisting of SEQ ID NO:16, SEQ ID NO:21, SEQ ID NO:26, SEQ ID NO:31, and SEQ ID NO:36, where each oligonucleotide probe among the collection includes a fluorophore moiety and a quencher moiety in energy transfer relationship with each other, where amplicons produced in the in vitro nucleic acid amplification reaction include the sequence of any of SEQ ID NO:13, SEQ ID NO:18, SEQ ID NO:23, SEQ ID NO:28, or SEQ ID NO:33 if the nucleic acid sample includes nucleic acids of macrolide-resistant M. genitalium, and where amplicons produced in the in vitro nucleic acid amplification reaction include the sequence of SEQ ID NO:11 if the nucleic acid sample includes nucleic acids of macrolide-sensitive M. genitalium. There further is the step of (b) detecting or having detected any of a fluorescent signal produced by the fluorophore moiety of one among the collection of oligonucleotides of the probe reagent in the in vitro nucleic acid amplification reaction, whereby if the fluorescent signal is detected then it is determined that the nucleic acid sample includes nucleic acids of macrolide-resistant M. genitalium, and whereby if the fluorescent signal is not detected then it is determined that the nucleic acid sample does not include nucleic acids of macrolide-resistant M. genitalium. In one preferred embodiment, the in vitro nucleic acid amplification reaction includes a primer extension step carried out at about 60° C. In some embodiments, the in vitro nucleic acid amplification reaction of step (a) is a polymerase chain reaction, and step (b) is performed as the polymerase chain reaction is occurring. In some embodiments, each of steps (a) and (b) is carried out using an automated nucleic acid analyzer instrument. In some embodiments, before step (a) there is a step for preparing the nucleic acid sample, or having the nucleic acid sample prepared, starting with a clinical specimen that may contain M. genitalium cellular material. For example, the step for preparing the nucleic acid sample, or for having the nucleic acid sample prepared, as well as steps (a) and (b) can be carried out using a single automated nucleic acid analyzer instrument. In some embodiments, the nucleic acid sample isolated from the specimen obtained from the human subject is known to include nucleic acids of M. genitalium before step (a) is conducted. In some embodiments, the method further includes the step of (c) treating the human subject based on the result of step (b). When it is determined in step (b) that the nucleic acid sample includes nucleic acids of macrolide-resistant M. genitalium, and step (c) includes treating the human subject with an antibiotic other than azithromycin. For example, the antibiotic other than azithromycin can be a fluoroquinolone antibiotic. In some embodiments, the nucleic acid sample isolated from the specimen obtained from the human subject is known to include nucleic acids of M. genitalium before step (a) is conducted, and it is determined in step (b) that the nucleic acid sample does not include nucleic acids of macrolide-resistant M. genitalium, and the method further includes the step of (c) treating the human subject with an antibiotic other than a fluoroquinolone antibiotic. In a preferred embodiment, the antibiotic other than the fluoroquinolone antibiotic is a macrolide antibiotic.

In another aspect, the disclosure relates to a probe for detecting nucleic acids of macrolide-resistant M. genitalium but not nucleic acids of macrolide-sensitive M. genitalium. Generally speaking, the probe includes an oligonucleotide up to 27 bases in length with 14 contiguous bases of SEQ ID NO:13, including position 11 of SEQ ID NO:13, allowing for substitution of RNA and DNA equivalent bases, and a detectable label covalently attached to the oligonucleotide. In a preferred embodiment, the oligonucleotide is up to 17 bases in length, and the oligonucleotide includes 14 contiguous bases of SEQ ID NO:14 or the complement thereof, allowing for substitution of RNA and DNA equivalent bases. In some embodiments, the oligonucleotide is up to 17 bases in length, and the oligonucleotide includes 14 contiguous bases of SEQ ID NO:14 or the complement thereof. In some embodiments, if the probe is included in a template-dependent nucleic acid amplification reaction having a primer and a DNA polymerase with 5′ to 3′ exonuclease activity, the oligonucleotide hydrolyzes during extension of the primer when the template being amplified includes the complement of SEQ ID NO:13, but not when the template being amplified includes the complement of SEQ ID NO:11. Preferably, the oligonucleotide hydrolyzes during extension of the primer at 60° C. when the template being amplified includes the complement of SEQ ID NO:13, but not when the template being amplified includes the complement of SEQ ID NO:11. In some embodiments, the detectable label includes a fluorophore moiety. When this is the case, the probe can further include a quencher moiety, where the quencher moiety is covalently attached to the oligonucleotide, and where the fluorophore moiety and the quencher moiety are in energy transfer relationship with each other. In some embodiments, the base sequence of the oligonucleotide is selected from the group consisting of SEQ ID NO:15, SEQ ID NO:16, and SEQ ID NO:17. In some embodiments, the fluorophore moiety is a fluorescein moiety covalently attached to the 5′-terminal nucleotide of the oligonucleotide, and the quencher moiety is covalently attached to the 3′-terminal nucleotide of the oligonucleotide. In a preferred embodiment, the base sequence of the probe is SEQ ID NO:16.

In another aspect, the disclosure relates to a probe for detecting nucleic acids of macrolide-resistant M. genitalium but not nucleic acids of macrolide-sensitive M. genitalium. Generally speaking, the probe can include: an oligonucleotide up to 27 bases in length with 15 contiguous bases of SEQ ID NO:18, including position 11 of SEQ ID NO:18, allowing for substitution of RNA and DNA equivalent bases, and a detectable label covalently attached to the oligonucleotide. In a preferred embodiment, the oligonucleotide is up to 18 bases in length, and the oligonucleotide includes 15 contiguous bases of SEQ ID NO:19 or the complement thereof, allowing for substitution of RNA and DNA equivalent bases. In some embodiments, the oligonucleotide is up to 18 bases in length, and the oligonucleotide includes 15 contiguous bases of SEQ ID NO:19 or the complement thereof. In some embodiments, if the probe is included in a template-dependent nucleic acid amplification reaction including a primer and a DNA polymerase with 5′ to 3′ exonuclease activity, the oligonucleotide hydrolyzes during extension of the primer when the template being amplified includes the complement of SEQ ID NO:18, but not when the template being amplified includes the complement of SEQ ID NO:11. Preferably, the oligonucleotide hydrolyzes during extension of the primer at 60° C. when the template being amplified includes the complement of SEQ ID NO:18, but not when the template being amplified includes the complement of SEQ ID NO:11. In some embodiments, the detectable label includes a fluorophore moiety. When this is the case, the probe can further include a quencher moiety, where the quencher moiety is covalently attached to the oligonucleotide, and where the fluorophore moiety and the quencher moiety are in energy transfer relationship with each other. In some embodiments, the base sequence of the oligonucleotide is selected from the group consisting of SEQ ID NO:20, SEQ ID NO:21, and SEQ ID NO:22. In some embodiments, the fluorophore moiety is a fluorescein moiety covalently attached to the 5′-terminal nucleotide of the oligonucleotide, and the quencher moiety is covalently attached to the 3′-terminal nucleotide of the oligonucleotide. In a preferred embodiment, the base sequence of the probe is SEQ ID NO:21.

In another aspect, the disclosure relates to a probe for detecting nucleic acids of macrolide-resistant M. genitalium but not nucleic acids of macrolide-sensitive M. genitalium. Generally speaking, the probe includes: an oligonucleotide up to 27 bases in length with 15 contiguous bases of SEQ ID NO:23, including position 11 of SEQ ID NO:23, allowing for substitution of RNA and DNA equivalent bases, and a detectable label covalently attached to the oligonucleotide. In a preferred embodiment, the oligonucleotide is up to 19 bases in length, and the oligonucleotide includes 15 contiguous bases of SEQ ID NO:24 or the complement thereof, allowing for substitution of RNA and DNA equivalent bases. In some embodiments, the oligonucleotide is up to 19 bases in length, and the oligonucleotide includes 15 contiguous bases of SEQ ID NO:24 or the complement thereof. In some embodiments, if the probe is included in a template-dependent nucleic acid amplification reaction including a primer and a DNA polymerase with 5′ to 3′ exonuclease activity, the oligonucleotide hydrolyzes during extension of the primer when the template being amplified includes the complement of SEQ ID NO:23, but not when the template being amplified includes the complement of SEQ ID NO:11. Preferably, the oligonucleotide hydrolyzes during extension of the primer at 60° C. when the template being amplified includes the complement of SEQ ID NO:23, but not when the template being amplified includes the complement of SEQ ID NO:11. In some embodiments, the detectable label includes a fluorophore moiety. When this is the case, the probe can further include a quencher moiety, where the quencher moiety is covalently attached to the oligonucleotide, and where the fluorophore moiety and the quencher moiety are in energy transfer relationship with each other. In some embodiments, the base sequence of the oligonucleotide is selected from the group consisting of SEQ ID NO:25, SEQ ID NO:26, and SEQ ID NO:27. In some embodiments, the fluorophore moiety is a fluorescein moiety covalently attached to the 5′-terminal nucleotide of the oligonucleotide, and the quencher moiety is covalently attached to the 3′-terminal nucleotide of the oligonucleotide. In some embodiments, the base sequence of the probe is SEQ ID NO:26.

In another aspect, the disclosure relates to a probe for detecting nucleic acids of macrolide-resistant M. genitalium but not nucleic acids of macrolide-sensitive M. genitalium. Generally speaking, the probe includes: an oligonucleotide up to 27 bases in length with 15 contiguous bases of SEQ ID NO:28, including position 12 of SEQ ID NO:28, allowing for substitution of RNA and DNA equivalent bases, and a detectable label covalently attached to the oligonucleotide. In a preferred embodiment, the oligonucleotide is up to 19 bases in length, and the oligonucleotide includes 15 contiguous bases of SEQ ID NO:29 or the complement thereof, allowing for substitution of RNA and DNA equivalent bases. In some embodiments, the oligonucleotide is up to 19 bases in length, and the oligonucleotide includes 15 contiguous bases of SEQ ID NO:29 or the complement thereof. In some embodiments, if the probe is included in a template-dependent nucleic acid amplification reaction including a primer and a DNA polymerase with 5′ to 3′ exonuclease activity, the oligonucleotide hydrolyzes during extension of the primer when the template being amplified includes the complement of SEQ ID NO:28, but not when the template being amplified includes the complement of SEQ ID NO:11. In a preferred embodiment, the oligonucleotide hydrolyzes during extension of the primer at 60° C. when the template being amplified includes the complement of SEQ ID NO:28, but not when the template being amplified includes the complement of SEQ ID NO: 11. In some embodiments, the detectable label includes a fluorophore moiety. When this is the case, the probe can further include a quencher moiety, where the quencher moiety is covalently attached to the oligonucleotide, and where the fluorophore moiety and the quencher moiety are in energy transfer relationship with each other. In some embodiments, the base sequence of the oligonucleotide is selected from the group consisting of SEQ ID NO:30, SEQ ID NO:31, and SEQ ID NO:32. In some embodiments, the fluorophore moiety is a fluorescein moiety covalently attached to the 5′-terminal nucleotide of the oligonucleotide, and the quencher moiety is covalently attached to the 3′-terminal nucleotide of the oligonucleotide. In a preferred embodiment, the base sequence of the probe is SEQ ID NO:31.

In another aspect, the disclosure relates to a probe for detecting nucleic acids of macrolide-resistant M. genitalium but not nucleic acids of macrolide-sensitive M. genitalium. Generally speaking, the probe can include: an oligonucleotide up to 27 bases in length with 15 contiguous bases of SEQ ID NO:33, including position 12 of SEQ ID NO:33, allowing for substitution of RNA and DNA equivalent bases, and a detectable label covalently attached to the oligonucleotide. In a preferred embodiment, the oligonucleotide is up to 18 bases in length, and the oligonucleotide includes 15 contiguous bases of SEQ ID NO:34 or the complement thereof, allowing for substitution of RNA and DNA equivalent bases. In some embodiments, the oligonucleotide is up to 18 bases in length, and the oligonucleotide includes 15 contiguous bases of SEQ ID NO:34 or the complement thereof. In some embodiments, if the probe is included in a template-dependent nucleic acid amplification reaction having a primer and a DNA polymerase with 5′ to 3′ exonuclease activity, the oligonucleotide hydrolyzes during extension of the primer when the template being amplified includes the complement of SEQ ID NO:33, but not when the template being amplified includes the complement of SEQ ID NO:11. In a preferred embodiment, the oligonucleotide hydrolyzes during extension of the primer at 60° C. when the template being amplified includes the complement of SEQ ID NO:33, but not when the template being amplified includes the complement of SEQ ID NO:11. In some embodiments, the detectable label includes a fluorophore moiety. When this is the case, the probe can further include a quencher moiety, where the quencher moiety is covalently attached to the oligonucleotide, and where the fluorophore moiety and the quencher moiety are in energy transfer relationship with each other. In some embodiments, the base sequence of the oligonucleotide is selected from the group consisting of SEQ ID NO:35, SEQ ID NO:36, and SEQ ID NO:37. In some embodiments, the fluorophore moiety is a fluorescein moiety covalently attached to the 5′-terminal nucleotide of the oligonucleotide, and the quencher moiety is covalently attached to the 3′-terminal nucleotide of the oligonucleotide. In a preferred embodiment, the base sequence of the probe is SEQ ID NO:36.

In another aspect, the disclosure relates to a probe reagent for detecting nucleic acids of macrolide-resistant M. genitalium. Generally speaking, the probe reagent includes: a collection of two or more oligonucleotide probes, where the base sequence of at least one oligonucleotide probe among the collection is selected from the group consisting of SEQ ID NO:16, SEQ ID NO:21, SEQ ID NO:26, SEQ ID NO:31, and SEQ ID NO:36, and where each oligonucleotide probe among the collection includes a fluorophore moiety and a quencher moiety in energy transfer relationship with each other. In a preferred embodiment, the base sequences of at least two oligonucleotide probes of the collection are selected from the group consisting of SEQ ID NO: 16, SEQ ID NO:21, SEQ ID NO:26, SEQ ID NO:31, and SEQ ID NO:36. In some embodiments, if the collection of oligonucleotide probes is included in a template-dependent nucleic acid amplification reaction including a primer and a DNA polymerase with 5′ to 3′ exonuclease activity, an oligonucleotide probe from among the collection hydrolyzes during extension of the primer when the template being amplified includes the complement of any of SEQ ID NO:13, SEQ ID NO:18, SEQ ID NO:23, SEQ ID NO:28, or SEQ ID NO:33, but not when the template being amplified includes the complement of SEQ ID NO:11. In a preferred embodiment, the oligonucleotide probe from among the collection hydrolyzes during extension of the primer at about 60° C. In some embodiments, the fluorophore moiety of each different oligonucleotide probe is attached to a terminal nucleotide thereof. In some embodiments, the fluorophore moiety is a fluorescein moiety. In some embodiments, the quencher moiety is the same for each of the oligonucleotide probes among the collection of two or more oligonucleotide probes.

### Definitions

Unless defined otherwise, all technical and scientific terms used herein have the same meaning as commonly understood by one of ordinary skill in the art pertinent to the methods and compositions described. General definitions may be found in technical books relevant to the art of molecular biology (e.g., Dictionary of Microbiology and Molecular Biology, 2nd ed., Singleton et al., 1994, John Wiley & Sons, New York, NY; or The Harper Collins Dictionary of Biology, Hale & Marham, 1991, Harper Perennial, New York, NY). As used herein, the following terms and phrases have the meanings ascribed to them unless specified otherwise.

The terms “a,” “an,” and “the” include plural referents, unless the context clearly indicates otherwise. For example, “a nucleic acid” as used herein is understood to represent one or more nucleic acids. As such, the terms “a” (or “an”), “one or more,” and “at least one” can be used interchangeably herein.

It will be appreciated that there is an implied “about” prior to the temperatures, concentrations, and times discussed in the present disclosure, such that slight and insubstantial deviations are within the scope of the present teachings. For example, conventional thermocycling instruments reach and maintain temperatures within a tolerance of ± 2° C., or even ± 1° C. Thus, “about” in the context of reaction temperatures means the specified temperature ± 2° C., or more preferably ± 1° C. With respect to reagent and component concentrations, “about” means ± 20%, or more preferably ± 10%. In general, the term “about” indicates insubstantial variation in a quantity of a component of a composition not having any significant effect on the activity or stability of the composition. All ranges are to be interpreted as encompassing the endpoints in the absence of express exclusions such as “not including the endpoints”; thus, for example, “within 10-15” includes the values 10 and 15.

Unless specifically noted, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). “Consisting essentially of” means that additional component(s), composition(s) or method step(s) that do not materially change the basic and novel characteristics of the compositions and methods described herein may be included in those compositions or methods. Such characteristics include the ability to detect a nucleic acid sequence present in a sample with specificity that distinguishes macrolide-resistant M. genitalium nucleic acid from macrolide-sensitive (i.e., wild-type) M. genitalium nucleic acid or other known pathogens, optionally at a sensitivity that can detect the target nucleic acid present in a sample at a concentration of about 50 copies/ml, and, optionally within about 60 minutes and/or within about 40 cycles from the beginning of an amplification reaction when a cycled amplification reaction is used.

As used herein, the term “sample” refers to a specimen that may contain macrolide-resistant M. genitialium or components thereof (e.g., nucleic acids). Samples may be from any source, such as biological specimens or environmental sources. Biological specimens include any tissue or material derived from a living or dead organism. Examples of biological samples include vaginal swab samples, respiratory tissue, exudates (e.g., bronchoalveolar lavage), biopsy, sputum, peripheral blood, plasma, serum, lymph node, gastrointestinal tissue, feces, urine, or other fluids, tissues or materials. Samples may be processed specimens or materials, such as obtained from treating a sample by using filtration, centrifugation, sedimentation, or adherence to a medium, such as matrix or support. Other processing of samples may include treatments to physically or mechanically disrupt tissue, cellular aggregates, or cells to release intracellular components that include nucleic acids into a solution which may contain other components, such as enzymes, buffers, salts, detergents, and the like. Samples being tested for the presence of an analyte may sometimes be referred to as “test samples.”

As used herein, a “nucleotide” is a subunit of a nucleic acid consisting of a phosphate group, a 5-carbon sugar, and a nitrogenous base (sometimes referred to as a “nucleobase”). The 5-carbon sugar found in RNA is ribose. In DNA, the 5-carbon sugar is 2′-deoxyribose. The term also includes analogs of such subunits, such as a methoxy group at the 2′ position of the ribose (also referred to herein as “2′-O-Me” or “2′-methoxy”).

“Nucleic acid” and “polynucleotide” refer to a multimeric compound comprising nucleosides or nucleoside analogs which have nitrogenous heterocyclic bases or base analogs linked together by a chemical backbone. The terms embrace conventional RNA, DNA, mixed RNA-DNA, and polymers that are analogs thereof. A nucleic acid “backbone” may be made up of a variety of linkages, including one or more of sugar-phosphodiester linkages, peptide-nucleic acid bonds (“peptide nucleic acids” or PNA; PCT Publication No. WO 95/32305), phosphorothioate linkages, methylphosphonate linkages, or combinations thereof. Sugar moieties of a nucleic acid may be ribose, deoxyribose, or similar compounds with substitutions (e.g., 2′ methoxy or 2′ halide substitutions). Nitrogenous bases may be conventional bases (A, G, C, T, U), analogs thereof (e.g., inosine or others; see The Biochemistry of the Nucleic Acids 5-36, Adams et al., ed., 11^{th} ed., 1992), derivatives of purines or pyrimidines (e.g., N⁴-methyl deoxyguanosine, deaza- or aza-purines, deaza- or aza-pyrimidines, pyrimidine bases with substituent groups at the 5 or 6 position, purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and 0⁴-alkyl-pyrimidines; U.S. Pat. No. 5,378,825 and PCT Publication No. WO 93/13121). Nucleic acids may include one or more “abasic” residues where the backbone includes no nitrogenous base for position(s) of the polymer (U.S. Pat. No. 5,585,481). A nucleic acid may comprise only conventional RNA or DNA sugars, bases and linkages, or may include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). Embodiments of oligomers that may affect stability of a hybridization complex include PNA oligomers, oligomers that include 2′-methoxy or 2′-fluoro substituted RNA, or oligomers that affect the overall charge, charge density, or steric associations of a hybridization complex, including oligomers that contain charged linkages (e.g., phosphorothioates) or neutral groups (e.g., methylphosphonates). 5-methylcytosines may be used in conjunction with any of the foregoing backbones/sugars/linkages including RNA or DNA backbones (or mixtures thereof) unless otherwise indicated. Similarly, 5-propynyl-2′-deoxycytidine (sometimes “pdC”) may be used in conjunction with any of the foregoing backbones/sugars/linkages including RNA or DNA backbones (or mixtures thereof) unless otherwise indicated. Likewise, 5-propynyl-2′-deoxyuridine (sometimes “pdU”) can be used as a substitute for “T” bases, and may be used in conjunction with any of the foregoing backbones/sugars/linkages including RNA or DNA backbones (or mixtures thereof) unless otherwise indicated. It is understood that when referring to ranges for the length of an oligonucleotide, amplicon, or other nucleic acid, that the range is inclusive of all whole numbers (e.g., 19-25 contiguous nucleotides in length includes 19, 20, 21, 22, 23, 24, and 25).

As used herein, an “oligonucleotide” (sometimes “oligomer” or “oligo”) is a molecule comprising two or more nucleotides (e.g., deoxyribonucleotides or ribonucleotides), preferably at least 5 nucleotides, more preferably at least about 10-15 nucleotides and more preferably at least about 15 to 30 nucleotides, or longer (e.g., oligonucleotides are typically less than 200 residues long (e.g., between 15 and 100 nucleotides). The exact size will depend on many factors, which in turn depend on the ultimate function or use of the oligonucleotide. Oligonucleotides are often referred to by their length. For example, a 24 residue oligonucleotide is referred to as a “24-mer.” Oligonucleotides can form secondary and tertiary structures by self-hybridizing or by hybridizing to other polynucleotides. Such structures can include, but are not limited to, duplexes, hairpins, cruciforms, bends, and triplexes. Oligonucleotides may be generated in any manner, including chemical synthesis, DNA replication, reverse transcription, PCR, or a combination thereof.

By “RNA and DNA equivalents” is meant RNA and DNA molecules having essentially the same complementary base pair hybridization properties. RNA and DNA equivalents have different sugar moieties (i.e., ribose versus deoxyribose) and may differ by the presence of uracil in RNA and thymine in DNA. The differences between RNA and DNA equivalents do not contribute to differences in homology because the equivalents have the same degree of complementarity to a particular sequence. By “DNA/RNA chimeric” is meant a nucleic acid comprising both DNA and RNA nucleotides. Unless the context clearly dictates otherwise, reference to an M. genitalium nucleic acid includes M. genitalium RNA and DNA equivalents, and DNA/RNA chimerics thereof.

By “RNA and DNA equivalent bases” is meant nucleotide bases having the same complementary base pair hybridization properties in RNA and DNA. Here the base uracil can be substituted in place of the base thymine, or vice versa, and so uracil and thymine are RNA and DNA equivalent bases. A polynucleotide base sequence 5′-AGCT-3′ that allows for substitution of RNA and DNA equivalent bases would also describe the sequence 5′-AGCU-3′. The differences between RNA and DNA equivalent bases do not contribute to differences in homology because the equivalents have the same degree of complementarity to a particular sequence.

The term, “complement” refers to a nucleic acid molecule that comprises a contiguous nucleic acid sequence that is complementary to a contiguous nucleic acid sequence of another nucleic acid molecule (for standard nucleotides A:T, A:U, C:G). For example, 5′-AACTGUC-3′ is the complement of 5′-GACAGTT-3′. Two nucleic acid sequences are “sufficiently complementary” when their respective contiguous nucleic acid sequences are at least 70% complementary.

A “target nucleic acid” as used herein is a nucleic acid comprising a target sequence to be amplified and/or detected. Target nucleic acids may be DNA or RNA, and may be either single-stranded or double-stranded. The target nucleic acid may include other sequences besides the target sequence, which may not be amplified.

The term “target sequence” as used herein refers to the particular nucleotide sequence of the target nucleic acid that is to be amplified and/or detected. The “target sequence” includes the complexing sequences to which oligonucleotides (e.g., primers) complex during an amplification processes (e.g., PCR, TMA). Where the target nucleic acid is originally single-stranded, the term “target sequence” will also refer to the sequence complementary to the “target sequence” as present in the target nucleic acid. Where the target nucleic acid is originally double-stranded, the term “target sequence” refers to both the sense (+) and antisense (-) strands.

“Target-hybridizing sequence” or “target-specific sequence” is used herein to refer to the portion of an oligomer that is configured to hybridize with a target nucleic acid sequence. Preferably, the target-hybridizing sequences are configured to specifically hybridize with a target nucleic acid sequence. Target-hybridizing sequences may be 100% complementary to the portion of the target sequence to which they are configured to hybridize, but not necessarily. Target-hybridizing sequences may also include inserted, deleted and/or substituted nucleotide residues relative to a target sequence.

The term “target a sequence,” as used herein in reference to a region of M. genitalium nucleic acid, refers to a process whereby an oligonucleotide hybridizes to a target sequence in a manner that allows for amplification and detection as described herein. In one preferred embodiment, the oligonucleotide is complementary to the targeted M. genitalium nucleic acid sequence and contains no mismatches. In another preferred embodiment, the oligonucleotide is complementary but contains 1, 2, 3, 4, or 5 mismatches with the targeted M. genitalium nucleic acid sequence. Preferably, the oligomer specifically hybridizes to the target sequence.

The term “configured to” denotes an actual arrangement of the polynucleotide sequence configuration of a referenced oligonucleotide target-hybridizing sequence. For example, amplification oligomers that are configured to generate a specified amplicon from a target sequence have polynucleotide sequences that hybridize to the target sequence and can be used in an amplification reaction to generate the amplicon. Also as an example, oligonucleotides that are configured to specifically hybridize to a target sequence have a polynucleotide sequence that specifically hybridizes to the referenced sequence under stringent hybridization conditions.

The term “configured to specifically hybridize to” as used herein means that the target-hybridizing region of an amplification oligonucleotide, detection probe, or other oligonucleotide is designed to have a polynucleotide sequence that could target a sequence of the referenced M. genitalium target region. Such an oligonucleotide is not limited to targeting that sequence only, but is rather useful as a composition, in a kit, or in a method for targeting an M. genitalium target nucleic acid. The oligonucleotide is designed to function as a component of an assay for amplification and detection of M. genitalium from a sample, and therefore is designed to target M. genitalium in the presence of other nucleic acids commonly found in testing samples. “Specifically hybridize to” does not mean exclusively hybridize to, as some small level of hybridization to non-target nucleic acids may occur. Rather, “specifically hybridize to” means that the oligonucleotide is configured to function in an assay to primarily hybridize the target so that an accurate detection of target nucleic acid in a sample can be determined.

The term “region,” as used herein, refers to a portion of a nucleic acid wherein said portion is smaller than the entire nucleic acid. For example, when the nucleic acid in reference is an amplicon, the term may be used to refer to the smaller nucleotide sequence identified for hybridization by the target-hybridizing sequence of a probe.

As used herein, the phrase “or its complement, or an RNA equivalent or DNA/RNA chimeric thereof,” with reference to a DNA sequence, includes (in addition to the referenced DNA sequence) the complement of the DNA sequence, an RNA equivalent of the referenced DNA sequence, an RNA equivalent of the complement of the referenced DNA sequence, a DNA/RNA chimeric of the referenced DNA sequence, and a DNA/RNA chimeric of the complement of the referenced DNA sequence.

Similarly, the phrase “or its complement, or a DNA equivalent or DNA/RNA chimeric thereof,” with reference to an RNA sequence, includes (in addition to the referenced RNA sequence) the complement of the RNA sequence, a DNA equivalent of the referenced RNA sequence, a DNA equivalent of the complement of the referenced RNA sequence, a DNA/RNA chimeric of the referenced RNA sequence, and a DNA/RNA chimeric of the complement of the referenced RNA sequence.

As used herein, a “primer” is an oligomer that hybridizes to a template nucleic acid and has a 3′ terminal hydroxyl group that can be extended by a polymerase (e.g., a DNA polymerase). A primer may be optionally modified (e.g., by including a 5′ region that is non-complementary to the target sequence). Such modification can include functional additions, such as tags, promoters, or other non-target-specific sequences used or useful for manipulating or amplifying the primer or target oligonucleotide.

“Nucleic acid amplification” refers to any in vitro procedure that produces multiple copies of a target nucleic acid sequence, or its complementary sequence, or fragments thereof (i.e., an amplified sequence containing less than the complete target nucleic acid). Examples of nucleic acid amplification procedures include the polymerase chain reaction (PCR) (e.g., U.S. Pat. Nos. 4,683,195, 4,683,202, and 4,800,159), ligase chain reaction (LCR) (e.g., EP Pat. No. 0320308), helicase-dependent amplification (e.g., U.S. Pat. No. 7,282,328), and strand-displacement amplification (SDA) (e.g., U.S. Pat. No. 5,422,252). Also included are replicase-mediated amplification (e.g., U.S. Pat. No. 4,786,600), and transcription associated methods, such as transcription-mediated amplification (TMA), nucleic acid sequence-based amplification (NASBA) and others (e.g., U.S. Pat. Nos. 5,399,491, 5,554,516, 5,437,990, 5,130,238, 4,868,105, and 5,124,246). Amplification may be linear or exponential. PCR amplification uses DNA polymerase, primers, and thermal cycling steps to synthesize multiple copies of the two complementary strands of DNA or cDNA. LCR amplification uses at least four separate oligonucleotides to amplify a target and its complementary strand by using multiple cycles of hybridization, ligation, and denaturation. Helicase-dependent amplification uses a helicase to separate the two strands of a DNA duplex generating single-stranded templates, followed by hybridization of sequence-specific primers hybridize to the templates and extension by DNA polymerase to amplify the target sequence. SDA uses a primer that contains a recognition site for a restriction endonuclease that will nick one strand of a hemi-modified DNA duplex that includes the target sequence, followed by amplification in a series of primer extension and strand displacement steps. Replicase-mediated amplification uses self-replicating RNA molecules, and a replicase such as QB-replicase. Particular embodiments use PCR or TMA, but it will be apparent to persons of ordinary skill in the art that oligomers disclosed herein may be readily used as primers in other amplification methods.

As used herein, the terms “polymerase chain reaction” and “PCR” refer to an enzymatic reaction in which a segment of DNA is replicated from a target nucleic acid in vitro. The reaction generally involves extension of a primer on each strand of a target nucleic acid with a template dependent DNA polymerase to produce a complementary copy of a portion of that strand. The chain reaction comprises iterative cycles of denaturation of the DNA strands, for example by heating, followed by cooling to allow primer annealing and extension, resulting in an exponential accumulation of copies of the region of the target nucleic acid that is flanked by and that includes the primer binding sites. When an RNA target nucleic acid is amplified by PCR, it is generally converted to a DNA copy strand with an enzyme capable of reverse transcription. Exemplary enzymes include MMLV reverse transcriptase, AMV reverse transcriptase, as well as other enzymes that will be familiar to those having an ordinary level of skill in the art.

By “amplicon” or “amplification product” is meant a nucleic acid molecule generated in a nucleic acid amplification reaction and which is derived from a target nucleic acid. An amplicon or amplification product contains a target nucleic acid sequence that may be of the same or opposite-sense as the target nucleic acid. Preferred amplification products comprise DNA.

As used herein, a “signal” is a detectable quantity or impulse of energy, such as electromagnetic energy (e.g., light). Emission of light from an appropriately stimulated fluorophore is an example of a fluorescent signal. In some embodiments, “signal” refers to the aggregated energy detected in a single channel of a detection instrument (e.g., a fluorometer).

As used herein, a “background” signal is the signal (e.g., a fluorescent signal) generated under conditions that do not permit a target nucleic acid-specific reaction (e.g., cleavage of a labeled oligonucleotide hydrolysis probe) to take place.

As used herein a “channel” of an energy sensor device, such as a device equipped with an optical energy sensor, refers to a pre-defined band of wavelengths that can be detected or quantified to the exclusion of other bands of wavelengths. For example, one detection channel of a fluorometer might be capable of detecting light energy emitted by one or more fluorescent labels over a range of wavelengths as a single event. Light emitted as the result of fluorescence can be quantified as relative fluorescence units (RFU) at a given wavelength, or over a band of wavelengths.

As used herein, the term “relative fluorescence unit” (“RFU”) is a unit of measurement of fluorescence intensity. RFU varies with the characteristics of the detection means used for the measurement, and can be used as a measurement to compare relative intensities between samples and controls.

As used herein, the term “detection probe” (or simply “probe”) refers to an oligomer that hybridizes specifically to a target sequence, including an amplified sequence, under conditions that promote nucleic acid hybridization, for detection of the target nucleic acid. Detection probes may be DNA, RNA, analogs thereof or combinations thereof (e.g., DNA/RNA chimerics), and they may be labeled or unlabeled. Detection probes may further include alternative backbone linkages (e.g., 2′-O-methyl linkages). A probe’s target sequence generally refers to the specific sequence within a larger sequence which the probe hybridizes specifically. A detection probe may include target-specific sequence(s) and non-target-specific sequence(s). Such non-target-specific sequences can include sequences which will confer a desired secondary or tertiary structure, such as a hairpin structure, which can be used to facilitate detection and/or amplification (see, e.g., U.S. Pat. Nos. 5,118,801, 5,312,728, 6,835,542, and 6,849,412). Probes of a defined sequence may be produced by techniques known to those of ordinary skill in the art, such as by chemical synthesis, and by in vitro or in vivo expression from recombinant nucleic acid molecules.

By “hybridization” or “hybridize” is meant the ability of two completely or partially complementary nucleic acid strands to come together (e.g., under specified hybridization assay conditions) in a parallel or antiparallel orientation to form a stable structure having a double-stranded region. The two constituent strands of this double-stranded structure, sometimes called a hybrid, are held together by hydrogen bonds. Although these hydrogen bonds most commonly form between nucleotides containing the bases adenine and thymine or uracil (A and T or U) or cytosine and guanine (C and G) on single nucleic acid strands, base pairing can also form between bases which are not members of these “canonical” pairs. Non-canonical base pairing is well-known in the art. See, e.g., R. L. P. Adams et al., The Biochemistry of the Nucleic Acids (11th ed. 1992).

By “preferentially hybridize” is meant that an amplification or detection probe oligomer can hybridize to its target nucleic acid to form stable oligomer:target hybrid, but not form a sufficient number of stable oligomer:non-target hybrids. Amplification and detection oligomers that preferentially hybridize to a target nucleic acid are useful to amplify and detect target nucleic acids, but not non-targeted organisms, especially phylogenetically closely related organisms. Thus, the oligomer hybridizes to target nucleic acid to a sufficiently greater extent than to non-target nucleic acid to enable one having ordinary skill in the art to accurately amplify and/or detect the presence (or absence) of nucleic acid derived from the specified target as appropriate. In general, reducing the degree of complementarity between an oligonucleotide sequence and its target sequence will decrease the degree or rate of hybridization of the oligonucleotide to its target region. However, the inclusion of one or more non-complementary nucleosides or nucleobases may facilitate the ability of an oligonucleotide to discriminate against non-target organisms. Preferential hybridization can be measured using techniques known in the art and described herein, such as in the examples provided below. In some embodiments, there is at least a 3-fold difference between target and non-target hybridization signals in a test sample, or at least a 5-fold difference between target and non-target hybridization signals in a test sample, or at least a 10-fold difference between target and non-target hybridization signals in a test sample, or at least a 100-fold difference, or at least a 1,000-fold difference. In some embodiments, non-target hybridization signals in a test sample are no more than the background signal level.

As used herein, “label” or “detectable label” refers to a moiety or compound attached or joined, directly or indirectly, to a probe that is detected or that leads to a detectable signal. Direct joining may use covalent bonds or non-covalent interactions (e.g., hydrogen bonding, hydrophobic or ionic interactions, and chelate or coordination complex formation) whereas indirect joining may use a bridging moiety or linker (e.g., via an antibody or additional oligonucleotide(s)). Any detectable moiety may be used, including a radionuclide, a ligand such as biotin or avidin or even a polynucleotide sequence, an enzyme, an enzyme substrate, a reactive group, a chromophore such as a dye or particle (e.g., a latex or metal bead) that imparts a detectable color, a luminescent compound (e.g., bioluminescent, phosphorescent, or a chemiluminescent compound), and a fluorescent compound or moiety (i.e., fluorophore). Embodiments of fluorophores include those that absorb light in the range of about 495 to 650 nm and emit light in the range of about 520 to 670 nm, which include those known as FAM™, TET™, CAL FLUOR™ (Orange or Red), and QUASAR™ compounds. Fluorophores may be used in combination with a quencher molecule that absorbs light when in close proximity to the fluorophore to diminish background fluorescence. Such quenchers are well known in the art and include, for example, BLACK HOLE QUENCHER™ (or BHQ™) or TAMRA™ compounds. Quencher moieties modified to include minor groove-binding (sometimes “MGB”) moieties are considered to be quenchers within the context of the disclosure.

Sequences are “sufficiently complementary” if they allow stable hybridization of two nucleic acid sequences, e.g., stable hybrids of probe and target sequences, although the sequences need not be completely complementary. That is, a “sufficiently complementary” sequence that hybridizes to another sequence by hydrogen bonding between a subset series of complementary nucleotides by using standard base pairing (e.g., G:C, A:T, or A:U), although the two sequences may contain one or more residues (including abasic positions) that are not complementary so long as the entire sequences in appropriate hybridization conditions to form a stable hybridization complex. Sufficiently complementary sequences may be at least about 80%, at least about 90%, or completely complementary in the sequences that hybridize together. Appropriate hybridization conditions are well-known to those skilled in the art, can be predicted based on sequence composition, or can be determined empirically by using routine testing (e.g., Sambrook et al., Molecular Cloning, A Laboratory Manual, 2^{nd} ed. at §§ 1.90-1.91, 7.37-7.57, 9.47-9.51 and 11.47-11.57, particularly §§ 9.50-9.51, 11.12-11.13, 11.45-11.47 and 11.55-11.57).

“Sample preparation” refers to any steps or method that treats a sample for subsequent amplification and/or detection of M. genitalium nucleic acids present in the sample. Samples may be complex mixtures of components of which the target nucleic acid is a minority component. Sample preparation may include any known method of concentrating components, such as microbes or nucleic acids, from a larger sample volume, such as by filtration of airborne or waterborne particles from a larger volume sample or by isolation of microbes from a sample by using standard microbiology methods. Sample preparation may include physical disruption and/or chemical lysis of cellular components to release intracellular components into a substantially aqueous or organic phase and removal of debris, such as by using filtration, centrifugation or adsorption. Sample preparation may include use of a nucleic acid oligonucleotide that selectively or non-specifically captures a target nucleic acid and separates it from other sample components (e.g., as described in US Pat. No. 6,110,678 and International Patent Application Pub. No. WO 2008/016988, each incorporated by reference herein).

“Separating” (and grammatical equivalents) or “purifying” (and grammatical equivalents) means that one or more components of a sample are removed or separated from other sample components. Sample components include target nucleic acids usually in a generally aqueous solution phase, which may also include cellular fragments, proteins, carbohydrates, lipids, and other nucleic acids. “Separating” or “purifying” does not connote any degree of purification. Typically, separating or purifying removes at least 70%, or at least 80%, or at least 95% of the target nucleic acid from other sample components.

The term “specificity,” in the context of an amplification and/or detection system, is used herein to refer to the characteristic of the system which describes its ability to distinguish between target and non-target sequences dependent on sequence and assay conditions. In terms of nucleic acid amplification, specificity generally refers to the ratio of the number of specific amplicons produced to the number of side-products (e.g., the signal-to-noise ratio). In terms of detection, specificity generally refers to the ratio of signal produced from target nucleic acids to signal produced from non-target nucleic acids.

The term “sensitivity” is used herein to refer to the precision with which a nucleic acid amplification reaction can be detected or quantitated. The sensitivity of an amplification reaction is generally a measure of the smallest copy number of the target nucleic acid that can be reliably detected in the amplification system, and will depend, for example, on the detection assay being employed, and the specificity of the amplification reaction, e.g., the ratio of specific amplicons to side-products.

A “reaction mixture” is a combination of reagents (e.g., oligonucleotides, target nucleic acids, enzymes, etc.) in a single reaction vessel.

As used herein, a “multiplex” assay is a type of assay that is able to detect or measure multiple analytes (e.g., two or more nucleic acid sequences) in a single run of the assay. It is distinguished from procedures that measure one analyte per reaction mixture. A multiplex assay can be carried out by combining into a single reaction vessel the reagents (e.g., probe reagents) for two or more different target sequences. In some embodiments, the same species of fluorescent reporter is detected in each of the assays of the multiplex.

As used herein, the term “donor” refers to a moiety (e.g., a fluorophore) that absorbs at a first wavelength and emits at a second, longer wavelength. The term “acceptor” refers to a moiety such as a fluorophore, chromophore, or quencher and that can absorb some or most of the emitted energy from the donor when it is near the donor group (e.g., between 1-100 nm). An acceptor may have an absorption spectrum that overlaps the donor’s emission spectrum. Generally, if the acceptor is a fluorophore, it then re-emits at a third, still longer wavelength; if it is a chromophore or quencher, it releases the energy absorbed from the donor without emitting a photon. In some preferred embodiments, alteration in energy levels of donor and/or acceptor moieties are detected (e.g., via measuring energy transfer, for example by detecting light emission) between or from donors and/or acceptor moieties). In some preferred embodiments, the emission spectrum of an acceptor moiety is distinct from the emission spectrum of a donor moiety such that emissions (e.g., of light and/or energy) from the moieties can be distinguished (e.g., spectrally resolved) from each other.

As used herein, “attached” (e.g., two things are “attached”) means chemically bonded together. For example, a fluorophore moiety is “attached” to an oligonucleotide probe when it is chemically bonded to the structure of the oligonucleotide probe.

As used herein, an “interactive” label pair refers to a donor moiety and an acceptor moiety (e.g., a quencher moiety) being attached to the same oligonucleotide probe, and being in energy transfer relationship (i.e., whether by a FRET or a non-FRET mechanism) with each other. A signal (e.g., a fluorescent signal) can be generated when the donor and acceptor moieties are separated, for example by hybridization and/or cleavage of a labeled oligonucleotide probe.

As used herein, emission from a donor moiety (e.g., a fluorophore) is “quenched” when the emission of a photon from the donor is prevented because an acceptor moiety (e.g., a quencher) is sufficiently close. For example, emission from a donor moiety is quenched when the donor moiety and the acceptor moiety are both attached to the same oligonucleotide probe.

The term “wild-type” (also “WT” herein) refers to a gene or gene product that has the characteristics of that gene or gene product when isolated from a common, naturally occurring source. In the context of the present disclosure, wild-type M. genitalium is macrolide-sensitive.

As used herein, a “threshold” or “threshold cutoff refers to a quantitative limit used for interpreting experimental results, where results above and below the cutoff lead to opposite conclusions. For example, a measured signal falling below a cutoff may indicate the absence of a particular target, but a measured signal that exceeds the same cutoff may indicate the presence of that target. By convention, a result that meets a cutoff (i.e., has exactly the cutoff value) is given the same interpretation as a result that exceeds the cutoff.

As used herein, a “threshold cycle number” refers to indicia of amplification that measure the time or cycle number when a real-time run curve signal crosses an arbitrary value or threshold. “TTime” and “Ct” determinations are examples of threshold-based indicia of amplification. Other methods involve performing a derivative analysis of the real-time run curve. For this disclosure, TArc and OTArc also can be used to determine when a real-time run curve signal crosses an arbitrary value (e.g., corresponding to a maximum or minimum angle in curvature, respectively). Methods of Time determination are disclosed in U.S. 8,615,368; methods of Ct determination are disclosed in EP 0640828 B1; derivative-based methods are disclosed in U.S. 6,303,305; and methods of TArc and OTArc determination are disclosed in U.S. 7,739,054. Those having an ordinary level of skill in the art will be aware of variations that also can be used for determining threshold cycle numbers.

As used herein, a “reaction vessel” or “reaction receptacle” is a container for holding a reaction mixture. Examples include individual wells of a multiwell plate, and plastic tubes (e.g., including individual tubes within a formed linear array of a multi-tube unit, etc.). However, it is to be understood that any suitable container may be used for containing the reaction mixture.

As used herein, a “vial” is a container, typically cylindrical, for holding liquid or dry (e.g., lyophilized) reagents. Vials commonly are used for packaging oligonucleotide or enzyme reagents into kits. Vials can be made of a variety of materials, such as glass or plastic.

### Introduction and Overview

Disclosed herein are oligonucleotides, compositions, kits, and methods that can be used to amplify and detect genetic markers of macrolide resistance in M. genitialium. While nucleic acids of wild-type (macrolide-sensitive) M. genitalium may be amplified, those sequences are not substantially detected by the labeled probes used to indicate macrolide resistance.

The disclosed method can be used for detecting and identifying macrolide-resistant M. genitialium by testing naive samples, but can also be used as a reflex assay that particularly reports the presence or absence of macrolide resistance in a sample already known to contain M. genitialium. The reflex assay approach can yield a superior positive predictive value for the assay. Positive predictive value correlates with prevalence. By testing a reflex sample set, the disclosed assay is used for testing samples known to be positive for M. genitalium, thereby maximizing the positive predictive value of the assay.

Procedures for identifying macrolide-resistant M. genitialium can be carried out in different ways. For example, there can be separate assays that independently identify the presence of nucleic acids characteristic of M. genitialium and the macrolide resistance marker (e.g., no shared oligonucleotides). Alternatively, standard microbiological culture techniques can be used to indicate the presence of M. genitialium in a sample that subsequently is tested for the presence of nucleic acid marker(s) of macrolide resistance. In some embodiments, a single assay can be used for detecting and identifying nucleic acid markers indicative of M. genitialium and macrolide resistance.

### DETAILED DESCRIPTION OF CERTAIN EMBODIMENTS

Disclosed is a technique that synthesizes multiple copies of an M. genitialium target nucleic acid and detects the sequences of macrolide-resistant variants. This can involve a pair of oligonucleotides, where one oligonucleotide is configured to hybridize to a sense strand of an M. genitalium nucleic acid and the other is configured to hybridize to an antisense strand of an M. genitalium nucleic acid. Such oligonucleotides include primer pairs for PCR or other forms of amplification. The amplification product (e.g., a PCR product) can include both wild-type sequence and sequence associated with resistance to macrolide antibiotics. In some embodiments, preferred probes that detect sequences indicating macrolide resistance do not also detect sequences associated with macrolide sensitivity. In some embodiments, the presence of wild-type M. genitalium 23S ribosomal nucleic acid sequences is determined using a different amplification product from the one used to establish the presence of nucleic acids harboring macrolide resistance markers.

As indicated above, the disclosed method or assay can be used as a reflex test to a positive result from a different assay that detects M. genitalium to determine if an infection with this organism is sensitive or resistant to azithromycin (a macrolide antibiotic). Stated differently, the disclosed method can be used for testing samples already known to contain M. genitialium bacteria. Patients identified as having azithromycin-resistant infections can be diverted to treatment with fluoroquinolones, the last known antibiotic class that is effective against M. genitialium.

The assay method can be carried out according to different assay formats. Optionally, M. genitialium-specific amplification products are detected at the end of an amplification reaction using an “end-point” formatted assay. Optionally, synthesis of M. genitialium-specific amplification products can be monitored periodically as the amplification reaction is taking place. This is sometimes referred to as a “real-time” formatted assay.

In some embodiments, one or more oligonucleotides, such as a primer set (defined as at least two primers configured to generate or detect an amplicon from a target sequence) or a primer set and an additional oligonucleotide (e.g., a probe) which is optionally non-extendible and/or labeled, are configured to hybridize to an amplification product of M. genitalium 23S ribosomal nucleic acid. In some embodiments, the primer set includes at least one reverse primer configured to hybridize to the 23S rRNA of M. genitalium, and at least one forward primer configured to hybridize to an extension product of the reverse primer using the ribosomal nucleic acid of M. genitalium as the template. When present, the additional oligonucleotide (e.g., a probe oligonucleotide) can be configured to hybridize to an amplicon produced by the primer set.

In some embodiments, a plurality of oligonucleotides, optionally non-extendible and/or labeled, are provided which collectively hybridize to one or more sequences within an M. genitalium nucleic acid amplification product. In some embodiments, a plurality of oligonucleotides, such as a plurality of primers or a plurality of primers and probes are provided which collectively hybridize to opposite strands of a double-stranded amplification product. In some embodiments, amplification or detection of the sequence indicative of M. genitalium discriminates the presence of M. genitalium from many other Mycoplasma species. Optionally, amplification or detection of the sequence indicative of M. genitalium can be highly specific for M. genitalium, so that nucleic acids from no other known organisms are detected.

In some embodiments, one or more oligonucleotides in a set, kit, composition, or reaction mixture include one or more methylated cytosine (e.g., 5-methylcytosine) residues. In some embodiments, at least about half of the cytosines in an oligonucleotide are methylated. In some embodiments, all or substantially all (e.g., all but one or two) of the cytosines in an oligonucleotide are methylated. For example, one or more cytosines at the 3′-end or within 2, 3, 4, or 5 bases of the 3′-end can be methylated. Alternatively, one or more cytosines at the 3′-end or within 2, 3, 4, or 5 bases of the 3′-end can be unmethylated.

In some embodiments, one or more oligonucleotides in a set, kit, composition, or reaction mixture include one or more 5-propynyl-modified cytidine (e.g., 5-propynyl-2′-deoxycytidine) residues. In some embodiments, at least about half of the cytidnes in an oligonucleotide are 5-propynylcytidine analogs. In some embodiments, all or substantially all (e.g., all but one or two) of the cytidines in an oligonucleotide are 5-propynylcytidine analogs. For example, one or more cytidines at the 3′-end or within 2, 3, 4, or 5 bases of the 3′-end can be 5-propynylcytidine analogs.

In some embodiments, one or more oligonucleotides in a set, kit, composition, or reaction mixture include one or more 5-propynyl-2′-deoxyuridine residues as substitutes for thymidine (“T”). In some embodiments, at least about half of the thymidines in an oligonucleotide are 5-propynyl-2′-deoxyuridine analogs. In some embodiments, all or substantially all (e.g., all but one or two) of the thymidines in an oligonucleotide are 5-propynyl-2′-deoxyuridine analogs. For example, one or more thymidines at the 3′-end or within 2, 3, 4, or 5 bases of the 3′-end can be 5-propynyl-2′-deoxyuridine analogs.

M. genitalium macrolide resistance can be assessed using reverse-transcription PCR of M. genitalium 23S rRNA, with hybridization probe-based detection to permit real-time monitoring of amplicon synthesis. To detect mutations at either of base locations 2058 or 2059 (E. coli numbering in region V of the 23S rRNA), which have been shown to be associated with M. genitalium macrolide resistance (see Couldwell et al., Infect. Drug Resist. 8:147-161 (2015)), a collection of probes was used. Macrolide resistance is indicated when there is a C or G at position 2059. Alternatively, macrolide resistance is indicated when the naturally occurring A residue at position 2058 is replaced by any of G, C, or T. Either of these conditions (i.e., mutation at one of two adjacent nucleotide positions) can result in macrolide resistance, and it is unnecessary for both positions to be mutated simultaneously to produce the drug-resistant condition. Optionally, each different base change indicative of macrolide resistance is detected using a different hybridization probe (e.g., a hydrolysis probe, useful in a TaqMan-formatted assay, labeled with each of a fluorophore and a quencher), where the detectable label is the same (e.g., the same fluorophore chemical species) for all probes. By this approach, macrolide resistance can be detected without identifying the position or identity of the base change leading to the antibiotic-resistant phenotype. In this way any genotype being associated with macrolide resistance can be indicated by a single type of fluorescent signal (e.g., a FAM signal).

In some embodiments, an oligonucleotide is provided that includes a label and/or is non-extendable. Such an oligonucleotide can be used as a probe or as part of a probe system. In some embodiments, the label is a non-nucleotide label. Example labels include compounds that emit a detectable light signal, such as fluorophores or luminescent (e.g., chemiluminescent) compounds that can be detected in a homogeneous mixture. More than one label, and more than one type of label, can be present on a particular probe, or detection can rely on using a mixture of probes in which each probe is labeled with a compound that produces a detectable signal (see e.g., U.S. Pat. Nos. 6,180,340 and 6,350,579). Labels can be attached to a probe by various means including covalent linkages, chelation, and ionic interactions. In some embodiments the label is covalently attached. For example, in some embodiments, a detection probe has an attached chemiluminescent label such as, for example, an acridinium ester (AE) compound (see e.g., U.S. Pat. Nos. 5,185,439; 5,639,604; 5,585,481; and 5,656,744). A label, such as a fluorescent or chemiluminescent label, can be attached to the probe by a non-nucleotide linker (see e.g., U.S. Pat. Nos. 5,585,481; 5,656,744; and 5,639,604).

In some embodiments, a probe can harbor two different labels (i.e., “first” and “second” labels), where the two labels interact with each other in an energy transfer relationship. These probes are sometimes referred to as “dual-label” probes. In one example, the first label can be a fluorescent moiety, and the second label can be a quencher moiety. Such probes can be used where hybridization of the probe to a target or amplicon followed by nucleolysis (i.e., hydrolysis of nucleic acid) by a polymerase including 5′-3′ exonuclease activity results in liberation of the fluorescent label and thereby increased fluorescence. This embraces the well known TaqMan™ assay format.

Examples of interacting donor/acceptor label pairs that can be used in connection with the disclosure include fluorescein/tetramethylrhodamine, IAEDANS/fluororescein, EDANS/DABCYL, coumarin/DABCYL, fluorescein/fluorescein, BODIPY® FL/BODIPY® FL, fluorescein/DABCYL, lucifer yellow/DABCYL, BODIPY®/DABCYL, eosine/DABCYL, erythrosine/DABCYL, tetramethylrhodamine/DABCYL, Texas Red/DABCYL, CY5/BHQ1®, CY5/BHQ2®, CY3/BHQ1(R), CY3/BHQ2® and fluorescein/QSY7® dye. Those having an ordinary level of skill in the art will understand that when donor and acceptor dyes are different, energy transfer can be detected by the appearance of sensitized fluorescence of the acceptor or by quenching of donor fluorescence. Non-fluorescent acceptors such as DABCYL and the QSY7® dyes advantageously eliminate the potential problem of background fluorescence resulting from direct (i.e., non-sensitized) acceptor excitation. Exemplary fluorophore moieties that can be used as one member of a donor-acceptor pair include fluorescein, HEX, ROX, and the CY dyes (such as CY5). Exemplary quencher moieties that can be used as another member of a donor-acceptor pair include DABCYL BLACKBERRY QUENCHER® which are available from Berry and Associates (Dexter, MI), and the BLACK HOLE QUENCHER® moieties which are available from Biosearch Technologies, Inc., (Novato, Calif.). One of ordinary skill in the art will be able to use appropriate pairings of donor and acceptor labels for use in various detection formats (e.g., FRET, TaqMan™, Invader®, etc.). Exemplified herein is the combination of a fluorescein (FAM) fluorescent donor moiety, and a BLACK HOLE QUENCHER® acceptor moiety.

Optionally, a probe oligonucleotide may be non-extendable. The oligonucleotide can be rendered non-extendable by the presence of a 3′-adduct (e.g., 3′-phosphorylation or 3′-alkanediol), having a 3′-terminal 3′-deoxynucleotide (e.g., a terminal 2′,3′-dideoxynucleotide), having a 3′-terminal inverted nucleotide (e.g., in which the last nucleotide is inverted such that it is joined to the penultimate nucleotide by a 3′ to 3′ phosphodiester linkage or analog thereof, such as a phosphorothioate), or having an attached fluorophore, quencher, or other label that interferes with extension (possibly but not necessarily attached via the 3′ position of the terminal nucleotide). In some embodiments, the 3′-terminal nucleotide is not methylated. In some embodiments, a detection oligonucleotide includes a 3′-terminal adduct such as a 3′-alkanediol (e.g., hexanediol).

In some embodiments, an oligonucleotide, such as a probe, is configured to specifically hybridize to an M. genitalium amplicon. The oligonucleotide can include or consist of a target-hybridizing sequence sufficiently complementary to the amplicon for specific hybridization. Optionally, the target-hybridizing sequence can be joined at its 5′-end to a nucleotide sequence that is not complementary to the amplicon being detected.

Also provided are kits for performing the methods described herein. “Kits” refer to packaged products that can be provided to an end-user, and typically will include one or more vials or containers holding one type of oligonucleotide, or a combination of different oligonucleotides or other reagents. A kit in accordance with the present disclosure can include at least one or more of the following: an amplification oligonucleotide, or oligonucleotide combination capable of amplifying an M. genitalium 23S ribosomal nucleic acid, or at least one detection probe as for determining the presence or absence of one or more macrolide resistance markers in an M. genitalium amplification product. In some embodiments, any oligonucleotide, or combination of oligonucleotides, described herein is present in the kit. The kits can further include a number of optional components such as, for example, capture probes (e.g., poly-(k) capture probes as described in US 2013/0209992), as well as a detectably labeled probe (e.g., a dual-labeled probe) that detects a wild-type M. genitalium sequence in an amplicon produced in the same reaction that amplified the macrolide resistance marker(s). To be clear, kits can include individual oligonucleotides or combinations of oligonucleotides in a single vial. Probe oligonucleotides, optionally including a detectable label (such as a fluorescent label), can be packaged individually, or can be packaged in combination with each other. Vials containing individual probes (e.g., each vial containing a different probe) optionally can be packaged into a single container, such as a box. Alternatively, kits can include one or more vials, where an individual vial contains a mixture of two or more different oligonucleotides (e.g., either primers and/or probes).

Other reagents that can be present in the kits include reagents suitable for performing in vitro amplification such as, for example, buffers, salt solutions, appropriate nucleotide triphosphates (e.g., dATP, dCTP, dGTP, and one or both of dTTP or dUTP; and/or ATP, CTP, GTP and UTP), and/or enzymes (e.g., a thermostable DNA polymerase, and/or reverse transcriptase and/or RNA polymerase and/or FEN enzyme), and will typically include test sample components, in which an M. genitalium target nucleic acid may or may not be present. In addition, for a kit that includes a detection probe together with an amplification oligonucleotide combination, selection of amplification oligonucleotides and detection probe oligonucleotides for a reaction mixture are linked by a common target region (i.e., the reaction mixture will include a probe that hybridizes to a sequence amplifiable by an amplification oligonucleotide combination of the reaction mixture). In certain embodiments, the kit further includes a set of instructions for practicing methods in accordance with the present disclosure, where the instructions can be associated with a package insert and/or the packaging of the kit or the components thereof.

Any method disclosed herein is also to be understood as a disclosure of corresponding uses of materials involved in the method directed to the purpose of the method. Any of the oligonucleotides including an M. genitalium sequence and any combinations (e.g., kits and compositions, including but not limited to reaction mixtures) including such an oligonucleotide are to be understood as also disclosed for use in detecting or quantifying macrolide-resistant M. genitalium, and for use in the preparation of a composition for detecting macrolide-resistant M. genitalium.

Broadly speaking, methods can employ one or more of the following elements: target capture, in which M. genitalium nucleic acid (e.g., from a sample, such as a clinical sample) is annealed to a capture oligonucleotide (e.g., a specific or nonspecific capture oligonucleotide); isolation (e.g., washing, to remove material not associated with a capture oligonucleotide); amplification; and amplicon detection, which for example can be performed in real-time with amplification. Certain embodiments involve each of the foregoing steps. Certain embodiments involve exponential amplification, optionally with a preceding linear amplification step. Certain embodiments involve exponential amplification and amplicon detection. Certain embodiments involve any two of the components listed above. Certain embodiments involve any two elements listed adjacently above (e.g., washing and amplification, or amplification and detection).

In some embodiments, amplification includes (1) contacting a nucleic acid sample with at least two oligonucleotides for amplifying a segment of M. genitalium 23S ribosomal nucleic acid, where the amplified segment includes positions corresponding to positions 2058 and 2059 of region V in E. coli 23S rRNA. The oligonucleotides can include at least two amplification oligonucleotides (e.g., one oriented in the sense direction and one oriented in the antisense direction for exponential amplification); (2) performing an in vitro nucleic acid amplification reaction, where any M. genitalium 23S ribosomal nucleic acid target present in the sample is used as a template for generating an amplification product; and (3) detecting the presence or absence of markers of macrolide resistance in the amplification product, thereby determining the presence or absence of macrolide-resistant M. genitalium in the sample. The markers of macrolide resistance include a C or G at position 2059, or a change from A to any of G, C, or T at position 2058.

Methods in accordance with the present disclosure can further include the step of obtaining the sample to be subjected to subsequent steps of the method. In certain embodiments, “obtaining” a sample to be used includes, for example, receiving the sample at a testing facility or other location where one or more steps of the method are performed, and/or retrieving the sample from a location (e.g., from storage or other depository) within a facility where one or more steps of the method are performed. Alternatively, the step of obtaining can involve lysing M. genitalium cells to release nucleic acids. Optionally, a target capture step for enhancement of M. genitalium rRNA can be included as a component of the obtaining step.

Exponentially amplifying a target sequence can utilize an in vitro amplification reaction using at least two amplification oligonucleotides that flank a target region to be amplified. In some embodiments, at least two amplification oligonucleotides as described above are provided. The amplification reaction can be temperature-cycled or isothermal. Suitable amplification methods include, for example, replicase-mediated amplification, polymerase chain reaction (PCR), ligase chain reaction (LCR), strand-displacement amplification (SDA), and transcription-mediated amplification (TMA).

A detection step can be performed using any of a variety of known techniques to detect a signal specifically associated with the amplified target sequence, such as by hybridizing the amplification product with a labeled detection probe and detecting a signal resulting from the labeled probe (including from label released from the probe following hybridization). In some embodiments, the labeled probe includes a second moiety, such as a quencher or other moiety that interacts with the first label, as discussed above. The detection step can also provide additional information on the amplified sequence, such as all or a portion of its nucleic acid sequence. Detection can be performed after the amplification reaction is completed, but preferably is performed simultaneously with amplifying the target region (e.g., in real-time). In one embodiment, the detection step allows homogeneous detection (e.g., detection of the hybridized probe without removal of unhybridized probe from the mixture (see e.g., U.S. Pat. Nos. 5,639,604 and 5,283,174)). In some embodiments, the nucleic acids are associated with a surface that results in a physical change, such as a detectable electrical change. Amplified nucleic acids can be detected by concentrating them in or on a matrix and detecting the nucleic acids or dyes associated with them (e.g., an intercalating agent such as ethidium bromide or SYBR® dye), or detecting an increase in dye associated with nucleic acid in solution phase. Other methods of detection can use nucleic acid detection probes configured to hybridize to a sequence in the amplified product and detecting the presence of the probe:product complex, or by using a complex of probes that can amplify the detectable signal associated with the amplified products (e.g., U.S. Pat. Nos. 5,424,413; 5,451,503; and 5,849,481; each incorporated by reference herein). Directly or indirectly labeled probes that specifically associate with the amplified product provide a detectable signal that indicates the presence of the target nucleic acid in the sample. In particular, the amplified product will contain a target sequence in or complementary to a sequence in the M. genitalium chromosome, and a probe will bind directly or indirectly to a sequence contained in the amplified product to indicate the presence of macrolide-resistant M. genitalium nucleic acid in the tested sample.

The disclosed assay can employ a target capture step as part of a procedure to obtain and isolate 23S rRNA from M. genitialium, then reverse transcription PCR with real-time detection to amplify and detect DNA copies of the 23S rRNA harboring marker(s) of macrolide resistance. A mixture of dual-labeled probes (e.g., labeled with a fluorophore and a quencher) can be used to interrogate base positions 2058 and 2059, which are mutated in M. genitialium that is resistant to macrolide antibiotics (e.g., azithromycin). Preferably, dual-labeled probes that produce signals indicating the presence of nucleic acid markers of macrolide resistance do not also produce signals indicating the presence of wild-type nucleic acids associated with macrolide-sensitive M. genitalium.

In some embodiments, a single fluorophore species produces signals indicating the presence of any of the macrolide resistance markers. Importantly, the disclosed technique can be used for detecting the genetic markers of macrolide resistance, without detecting wild-type sequences, even among a background of wild-type M. genitialium sequences that may be present in a mixed infection.

Briefly, the target capture method used in the presently disclosed assay can employ an oligonucleotide probe immobilized directly to a magnetically attractable solid support (i.e., the “immobilized probe”) and a “capture probe” (or sometimes “target capture probe” or “target capture oligonucleotide”) that bridged the immobilized probe and the 23S M. genitialium target ribosomal nucleic acid to form a hybridization complex that could be separated from other components in the mixture. An illustrative instrument workstation that can be used to carry out such a purification step is disclosed by Acosta et al., in U.S. Pat. No. 6,254,826, the disclosure of which is incorporated by reference. The capture probe is preferably designed so that the melting temperature of the capture probe:target nucleic acid hybrid is greater than the melting temperature of the capture probe: immobilized probe hybrid. In this way, different sets of hybridization assay conditions can be employed to facilitate hybridization of the capture probe to the target nucleic acid prior to hybridization of the capture probe to the immobilized oligonucleotide, thereby maximizing the concentration of free probe and providing favorable liquid phase hybridization kinetics. This “two-step” target capture method is disclosed by Weisburg et al., U.S. Pat. No. 6,110,678. In some embodiments, the 23S M. genitalium target ribosomal nucleic acid is captured onto the solid support by direct interaction (e.g., hybridization) with the immobilized probe, and there is no requirement for a target capture probe. Other target capture schemes readily adaptable to the present technique are well known in the art and include, without limitation, those disclosed by the following: Dunn et al., Methods in Enzymology, “Mapping viral mRNAs by sandwich hybridization,” 65(1):468-478 (1980); Ranki et al., U.S. Pat. No. 4,486,539; Stabinsky, U.S. Pat. No. 4,751,177; and Becker et al., U.S. Pat. No. 6,130,038.

Isolation can follow capture, wherein the complex on the solid support is separated from other sample components. Isolation can be accomplished by any appropriate technique (e.g., washing a support associated with the M. genitalium target sequence one or more times (e.g., 2 or 3 times) to remove other sample components and/or unbound oligonucleotide). In embodiments using a particulate solid support, such as paramagnetic beads, particles associated with the M. genitalium-target can be suspended in a washing solution and retrieved from the washing solution, in some embodiments by using magnetic attraction. To limit the number of handling steps, the M. genitalium target nucleic acid can be amplified by simply mixing the M. genitalium target sequence in the complex on the support with amplification oligonucleotides and proceeding with amplification steps.

### Methods of Treatment and Changing Treatments

In some embodiments, the assays disclosed herein can be selected or ordered from a menu of testing options available to a healthcare professional caring for a human patient. For example, a physician may place an order using an electronic, paper, or other ordering system so that a sample obtained from the human patient will be subjected to the various steps needed to determine the presence or absence of macrolide-sensitive M. genitalium and/or of macrolide-resistant M. genitalium. In this regard, the individual placing the order or request can be said to “direct” or “have” certain steps performed for the purpose of making the determination regarding the presence or absence of the M. genitalium organism (e.g., the macrolide-resistant organism). For example, there can be a step for obtaining, or “having” obtained the sample to be used for testing, etc. Simply stated, the individual requesting an assay need not perform all of the procedural steps themselves. Of course, this might be considered relevant not only for initiating the sequence of events needed to obtain the molecular diagnostic result, but also relevant for automated systems, or data processing systems where data analysis is performed at a remote site.

In some embodiments, the molecular diagnostic assay is useful for detecting the presence of wildtype M. genitalium, and of determining the macrolide-resistance status of the organism, if present in the test sample. For example, a single test may combine detection of genetic markers for M. genitalium (e.g., the wildtype organism) and for macrolide-resistance. In a different embodiment, the assay for detecting macrolide-resistance can be performed on a test sample that previously was determined by independent testing to contain M. genitalium. This latter approach is sometimes referred to as a “reflex” test.

If it is determined that an M. genitalium-containing test sample obtained from a patient either includes or does not include macrolide-resistant M. genitalium, then a course of action can be implemented or changed to treat the patient for an improved outcome. If it is determined that the sample obtained from the patient includes macrolide-resistant M. genitalium, then the patient can be treated with a course of one or more antibiotics other than a macrolide antibiotic (e.g., azithromycin). For example, the treating healthcare professional may elect to prescribe, recommend, or treat with a fluoroquinolone antibiotic, or another agent effective against macrolide-resistant M. genitalium. Alternatively, if it is determined that the patient sample includes nucleic acids of M. genitalium, but does not include nucleic acids of macrolide-resistant M. genitalium, then a course of antibiotics other than fluoroquinolones may be prescribed or recommended. For example, a patient harboring an infection with M. genitalium that is not macrolide-resistant M. genitalium may be treated with a macrolide antibiotic (e.g., azithromycin) or another antibiotic effective against M. genitalium. Yet a different possibility is that a patient may have been treated with a course of fluoroquinolone antibiotics that will have been effective at controlling or eliminating an infection with macrolide-resistant M. genitalium. A subsequent test result indicating the absence of macrolide-resistant M. genitalium nucleic acid in a sample obtained following the initial treatment may guide the healthcare professional to change the treatment plan by discontinuing administration of the fluoroquinolone antibiotic (e.g., because it is no longer necessary).

### Illustrative Examples

Template nucleic acids to be amplified included the sequences of SEQ ID Nos: 1-6 or the complements thereof, allowing for substitution of RNA and DNA equivalent bases. In one embodiment, the wild-type template included the sequence complementary to SEQ ID NO:1, where positions 73 and 74 of SEQ ID NO:1 correspond to positions referenced herein as 2058 and 2059, respectively. Bases located in the wild-type template at both of these positions in SEQ ID NO:1 (i.e., corresponding to base positions 73 and 74, respectively) are A residues. Macrolide resistance is indicated when referenced position 2058, which corresponds to position 73 in SEQ ID NO:1, is occupied by any of C (e.g., “2058C” appearing in SEQ ID NO:2), G (e.g., “2058G” appearing in SEQ ID NO:3) or T (e.g., “2058T” appearing in SEQ ID NO:4). Macrolide resistance also is indicated when referenced position 2059, which corresponds to position 74 in SEQ ID NO:1, is occupied by either C (e.g., “2059C” appearing in SEQ ID NO:5) or G (e.g., “2059G” appearing in SEQ ID NO:6).

Preferred primers useful for amplifying any of the template nucleic acids can be 15 to 30 bases in length, and can include at least 15 contiguous bases of SEQ ID NO:7, or at least 15 contiguous bases of SEQ ID NO:9. An exemplary reverse primer had the sequence of SEQ ID N0:10, while an exemplary forward primer had the sequence of SEQ ID NO:8. Amplification products produced using the wild-type template of SEQ ID NO: 1 included SEQ ID NO:11 or the complement thereof. Here base positions 11 and 12 of SEQ ID NO:11 corresponded to positions referenced herein as 2058 and 2059, respectively. Preferably, hybridization probes useful for detecting nucleic acids characteristic of macrolide resistance do not produce detectable signals resulting from binding to the wild-type amplification product comprising the sequence of SEQ ID NO:11 or the complement thereof during real-time nucleic acid amplification reactions.

Amplification products characteristic of macrolide-resistant M. genitalium included the sequence of SEQ ID NO:12 or the complement thereof, where base positions 11 and 12 of SEQ ID NO:12 corresponded to positions referenced herein as 2058 and 2059, respectively. Referring to SEQ ID NO:12, macrolide resistance was indicated when either position 11 was occupied by C, G, or T; or when position 12 was occupied by C or G.

Generally speaking, the 2058C amplification product characteristic of macrolide resistance includes SEQ ID NO:13 or the complement thereof. This amplification product can be detected using a probe, preferably up to 27 bases in length, having at least 14 contiguous bases of SEQ ID NO:13, and including position 11 of SEQ ID NO:13, or the complements of these sequences, allowing for substitution of RNA and DNA equivalent bases. In one preferred embodiment, the 2058C amplification product complementary to SEQ ID NO:13 is detected using a probe up to 27 bases in length, having at least 14 contiguous bases of SEQ ID NO:13. Optionally, the probe includes at least one detectable label (e.g., a fluorophore). In some embodiments, the probe includes a fluorophore (e.g., a fluorophore joined to the 5′ terminal nucleotide) and a quencher (e.g., a quencher joined to the 3′ terminal nucleotide), where the fluorophore and quencher are in energy transfer relationship with each other. Still more preferably, the probe is up to 17 bases in length, and includes 14 contiguous bases of SEQ ID NO:14 or the complement thereof, allowing for substitution of RNA and DNA equivalent bases. Yet more preferably, the probe is up to 16 bases in length, and includes 14 contiguous bases of SEQ ID NO:14 or the complement thereof, allowing for substitution of RNA and DNA equivalent bases. Exemplary probes having these features include SEQ ID NO:15, SEQ ID NO:16, and SEQ ID NO:17. Preferably, labeled probes that detect the 2058C amplification product including SEQ ID NO:13 or the complement thereof do not also detect the amplified wild-type sequence that includes SEQ ID NO:11 or the complement thereof.

Generally speaking, the 2058G amplification product characteristic of macrolide resistance includes SEQ ID NO:18 or the complement thereof. This amplification product can be detected using a probe, preferably up to 27 bases in length, having at least 15 contiguous bases of SEQ ID NO:18, and including position 11 of SEQ ID NO:18, or the complements of these sequences, allowing for substitution of RNA and DNA equivalent bases. In one preferred embodiment, the 2058G amplification product complementary to SEQ ID NO:18 is detected using a probe up to 27 bases in length, having at least 15 contiguous bases of SEQ ID NO:18. Optionally, the probe includes at least one detectable label (e.g., a fluorophore). In some embodiments, the probe includes a fluorophore (e.g., a fluorophore joined to the 5′ terminal nucleotide) and a quencher (e.g., a quencher joined to the 3′ terminal nucleotide), where the fluorophore and quencher are in energy transfer relationship with each other. Still more preferably, the probe is up to 18 bases in length, and includes 15 contiguous bases of SEQ ID NO:19 or the complement thereof, allowing for substitution of RNA and DNA equivalent bases. Yet more preferably, the probe is up to 16 bases in length, and includes 15 contiguous bases of SEQ ID NO:19 or the complement thereof, allowing for substitution of RNA and DNA equivalent bases. Exemplary probes having these features include SEQ ID NO:20, SEQ ID NO:21, and SEQ ID NO:22. Preferably, labeled probes that detect the 2058G amplification product including SEQ ID NO:18 or the complement thereof do not also detect the amplified wild-type sequence that includes SEQ ID NO:11 or the complement thereof.

Generally speaking, the 2058T amplification product characteristic of macrolide resistance includes SEQ ID NO:23 or the complement thereof. This amplification product can be detected using a probe, preferably up to 27 bases in length, having at least 15 contiguous bases of SEQ ID NO:23, and including position 11 of SEQ ID NO:23, or the complements of these sequences, allowing for substitution of RNA and DNA equivalent bases. In one preferred embodiment, the 2058T amplification product complementary to SEQ ID NO:23 is detected using a probe up to 27 bases in length, having at least 15 contiguous bases of SEQ ID NO:23. Optionally, the probe includes at least one detectable label (e.g., a fluorophore). In some embodiments, the probe includes a fluorophore (e.g., a fluorophore joined to the 5′ terminal nucleotide) and a quencher (e.g., a quencher joined to the 3′ terminal nucleotide), where the fluorophore and quencher are in energy transfer relationship with each other. Still more preferably, the probe is up to 19 bases in length, and includes 15 contiguous bases of SEQ ID NO:24 or the complement thereof, allowing for substitution of RNA and DNA equivalent bases. Yet more preferably, the probe is up to 16 bases in length, and includes 15 contiguous bases of SEQ ID NO:24 or the complement thereof, allowing for substitution of RNA and DNA equivalent bases. Exemplary probes having these features include SEQ ID NO:25, SEQ ID NO:26, and SEQ ID NO:27. Preferably, labeled probes that detect the 2058T amplification product including SEQ ID NO:23 or the complement thereof do not also detect the amplified wild-type sequence that includes SEQ ID NO:11 or the complement thereof.

Generally speaking, the 2059C amplification product characteristic of macrolide resistance includes SEQ ID NO:28 or the complement thereof. This amplification product can be detected using a probe, preferably up to 27 bases in length, having at least 15 contiguous bases of SEQ ID NO:28, and including position 12 of SEQ ID NO:28, or the complements of these sequences, allowing for substitution of RNA and DNA equivalent bases. In one preferred embodiment, the 2059C amplification product complementary to SEQ ID NO:28 is detected using a probe up to 27 bases in length, having at least 15 contiguous bases of SEQ ID NO:28. Optionally, the probe includes at least one detectable label (e.g., a fluorophore). In some embodiments, the probe includes a fluorophore (e.g., a fluorophore joined to the 5′ terminal nucleotide) and a quencher (e.g., a quencher joined to the 3′ terminal nucleotide), where the fluorophore and quencher are in energy transfer relationship with each other. Still more preferably, the probe is up to 19 bases in length, and includes 15 contiguous bases of SEQ ID NO:29 or the complement thereof, allowing for substitution of RNA and DNA equivalent bases. Yet more preferably, the probe is up to 16 bases in length, and includes 15 contiguous bases of SEQ ID NO:29 or the complement thereof, allowing for substitution of RNA and DNA equivalent bases. Exemplary probes having these features include SEQ ID NO:30, SEQ ID NO:31, and SEQ ID NO:32. Preferably, labeled probes that detect the 2059C amplification product including SEQ ID NO:28 or the complement thereof do not also detect the amplified wild-type sequence that includes SEQ ID NO:11 or the complement thereof.

Generally speaking, the 2059G amplification product characteristic of macrolide resistance includes SEQ ID NO:33 or the complement thereof. This amplification product can be detected using a probe, preferably up to 27 bases in length, having at least 15 contiguous bases of SEQ ID NO:33, and including position 12 of SEQ ID NO:33, or the complements of these sequences, allowing for substitution of RNA and DNA equivalent bases. In one preferred embodiment, the 2059G amplification product complementary to SEQ ID NO:33 is detected using a probe up to 27 bases in length, having at least 15 contiguous bases of SEQ ID NO:33. Optionally, the probe includes at least one detectable label (e.g., a fluorophore). In some embodiments, the probe includes a fluorophore (e.g., a fluorophore joined to the 5′ terminal nucleotide) and a quencher (e.g., a quencher joined to the 3′ terminal nucleotide), where the fluorophore and quencher are in energy transfer relationship with each other. Still more preferably, the probe is up to 18 bases in length, and includes 15 contiguous bases of SEQ ID NO:34 or the complement thereof, allowing for substitution of RNA and DNA equivalent bases. Yet more preferably, the probe is up to 16 bases in length, and includes 15 contiguous bases of SEQ ID NO:34 or the complement thereof, allowing for substitution of RNA and DNA equivalent bases. Exemplary probes having these features include SEQ ID NO:35, SEQ ID NO:36, and SEQ ID NO:37. Preferably, labeled probes that detect the 2059G amplification product including SEQ ID NO:33 or the complement thereof do not also detect the amplified wild-type sequence that includes SEQ ID NO:11 or the complement thereof.

The following Examples illustrate certain embodiments of the assay technique, and are not to be construed as limiting the scope of the disclosure in any way. The procedures and oligonucleotide reagents illustrate detection of macrolide resistance (e.g., azithromycin resistance) markers involving a single nucleotide substitution mutation located either at position 2058 (C/G/T substitution) or 2059 (C/G substitution) of the 23S rRNA gene sequence (positions in E. coli annotation are 2071 and 2072). Optionally, urine or urogenital swab samples can serve as the source of nucleic acids to be tested using any of the oligonucleotide probes or reaction mixtures (e.g., individual probes, or combinations of probes and/or primers) described below. To demonstrate the technique in a manner that permitted rigorous sensitivity testing, in vitro transcripts (IVTs) were used as model target nucleic acids. The IVT harboring the wild-type M. genitalium 23S ribosomal nucleic acid sequence was synthesized from a template that included the sequence of SEQ ID NO: 1 and the complement thereof (i.e., the ITV being the RNA equivalent of a portion of SEQ ID NO:1). The IVT harboring the 2058C macrolide resistance mutation was synthesized from a template that included the sequence of SEQ ID NO:2 and the complement thereof (i.e., the ITV being the RNA equivalent of a portion of SEQ ID NO:2). The IVT harboring the 2058G macrolide resistance mutation was synthesized from a template that included the sequence of SEQ ID NO:3 and the complement thereof (i.e., the ITV being the RNA equivalent of a portion of SEQ ID NO:3). The IVT harboring the 2058T macrolide resistance mutation was synthesized from a template that included the sequence of SEQ ID NO:4 and the complement thereof (i.e., the ITV being the RNA equivalent of a portion of SEQ ID NO:4). The IVT harboring the 2059C macrolide resistance mutation was synthesized from a template that included the sequence of SEQ ID NO:5 and the complement thereof (i.e., the ITV being the RNA equivalent of a portion of SEQ ID NO:5). The IVT harboring the 2059G macrolide resistance mutation was synthesized from a template that included the sequence of a portion of SEQ ID NO:6 and the complement thereof (i.e., the ITV being the RNA equivalent of SEQ ID NO:6).

The same primer set was used for amplifying the different templates harboring drug resistance markers in all of the procedures disclosed below. Determined Ct (i.e., cycle threshold) values indicated the cycle number at which a detectable amplification signal met a threshold value representing a predetermined level of reaction progress. Ct values below 45 cycles were regarded as indicating positive results or “calls” (i.e., target nucleic acid was present in the sample undergoing testing).

Example 1 describes early procedures that detected single nucleotide substitution mutations characteristic of macrolide resistance in M. genitalium.

### Example 1

### Multiplex Amplification and Det×ection of Macrolide Resistance Markers in M. Genitalium 23S rRNA

The Panther Fusion System (Gen-Probe Incorporated; San Diego, CA) for automated nucleic acid analysis was employed for amplifying 23S ribosomal nucleic acid sequences of M. genitalium using the polymerase chain reaction, with monitoring of amplicon production as reaction cycles were occurring (i.e., a real-time PCR format). Samples used as sources of amplifiable templates included either 500 copies/reaction or 50 copies/reaction of an IVT corresponding to one of the five macrolide-resistant M. genitalium 23S rRNA sequences. Control reactions used to confirm specificity of the 5-plex macrolide resistance assays were performed using 23S rRNA templates isolated from 1 × 10⁵ CFU/ml of wild-type (macrolide-sensitive) M. genitalium. The fact that no non-specific signal was observed in the FAM channel for the negative controls confirmed the assays were specific for detection of macrolide resistance markers. An internal control (IC) RNA template, together with primers and a probe for amplifying and detecting the IC also were included. Template nucleic acids were enriched by target capture onto magnetic beads before being combined with enzymes, dNTPs, and cofactors in reaction mixtures that supported reverse transcription and PCR amplification, as will be familiar to those having an ordinary level of skill in the art. Replicates of multiplex reaction mixtures included forward and reverse primers, together with all five labeled oligonucleotide probes. Probes were labeled with a fluorescent dye at the 5′-end, and with a quencher moiety at the 3′-end. In all instances the fluorescent label was fluorescein. The quencher moiety was the commercially available Black Hole Quencher® fluorescent energy transfer dye (Biosearch Technologies, Inc.; Petaluma, CA). Reaction conditions included: 8 minutes at 46° C. for reverse transcription to synthesize cDNA; a 2 minute 95° C. activation step; 45 cycles of 5 seconds at 95° C. to denature double stranded nucleic acids, and 22 seconds at 60° C. for primer annealing and extension. Amplicon synthesis was monitored by detecting fluorescence signals in the FAM channel as a function of cycle number. Sequences of relevant oligonucleotide reagents used in the procedure are presented in Table 1.

TABLE 1

Oligonucleotide Reagents

| | | |
|---|---|---|
| Oligo Name | Oligo Function | Oligo Sequence |
| SEQ ID NO:8 | Forward primer | CTCGGTGAAATCCAGGTACG |
| SEQ ID NO:10 | Reverse primer | GTATTCCACATTTCACATCAACAAA |
| SEQ ID NO:15 | 2058C Probe | GACGGCAAGACCCC |
| SEQ ID NO:20 | 2058G Probe | GGACGGGAAGACCCC |
| SEQ ID NO:25 | 2058T Probe | GGGACGGTAAGACCCC |
| SEQ ID NO:30 | 2059C Probe | GGGACGGACAGACCCC |
| SEQ ID NO:35 | 2059G Probe | GGACGGAGAGACCCC |

The results presented in Table 2 demonstrated that each of five IVT templates harboring a marker of macrolide resistance was detectable in the multiplex formatted assay. The first column in the table identifies the IVT containing a single nucleotide substitution characteristic of macrolide resistance in M. genitalium. Tabulated results show that analytical sensitivity for trials conducted using 50 copies/reaction of the IVTs was weaker (Ct values approached 45 cycles) than desired, and was not balanced. For example, at an input level of 50 copies/reaction positive detection of the 2059G target was achieved 60% of the time, but positive detection of the 2058T target was achieved only 30% of the time. Although not shown, specificity of each assay for detecting macrolide resistance was confirmed by the absence of a specific fluorescent signal in the FAM channel when the wild-type M. genitalium 23S rRNA IVT was used as a template. Amplification of an internal control template nucleic acid was detected in each reaction mixture, thereby confirming integrity of the amplification and detection procedure.

TABLE 2

Real-Time Amplification Results

| In vitro transcript | 500 copies/PCR | | 50 copies/PCR | |
|---|---|---|---|---|
| | Mean Ct | Pos call % | Mean Ct | Pos call % |
| 2058C | 42.50 | 100 | 43.80 | 40 |
| 2058G | 41.98 | 100 | 43.57 | 40 |
| 2058T | 43.29 | 100 | 44.60 | 30 |
| 2059C | 39.61 | 100 | 42.16 | 50 |
| 2059G | 42.25 | 100 | 44.00 | 60 |

Example 2 describes testing that explored improvements to enhance assay sensitivity. Dual-labeled probes in this Example were either 15 or 16 nucleotides in length, and were used individually (i.e., in singleplex reactions) rather than as combinations (i.e., in multiplex reactions).

### Example 2

### Singleplex Amplification and Detection of Macrolide Resistance Markers in M. Genitalium 23S rRNA

The Panther Fusion System for automated nucleic acid analysis was again employed to amplify 23S ribosomal nucleic acid sequences of M. genitalium using the polymerase chain reaction, with monitoring of amplicon production as reaction cycles were occurring. Samples used as sources of amplifiable templates included either 500 copies/reaction or 50 copies/reaction of an IVT corresponding to one of the five macrolide-resistant M. genitalium 23S rRNA sequences. As in Example 1, control reactions used to confirm specificity of the macrolide resistance assays were performed using 23S rRNA templates isolated from 1 × 10⁵ CFU/ml of wild-type (macrolide-sensitive) M. genitalium. An IC RNA template, together with primers and a probe for amplifying and detecting the IC template also were included. Template nucleic acids were enriched by target capture onto magnetic beads before being combined with enzymes, dNTPs, and cofactors in reaction mixtures that supported reverse transcription and PCR amplification, as will be familiar to those having an ordinary level of skill in the art. Replicates of singleplex reaction mixtures included the forward and reverse primers from Example 1, together with one of the oligonucleotide probes presented in Table 3. Probes were labeled with a fluorescent dye at the 5′-end, and with a quencher moiety at the 3′-end, also as described under Example 1. Reaction conditions included: 8 minutes at 46° C. for reverse transcription to synthesize cDNA; a 2 minute 95° C. activation step; 45 cycles of 5 seconds at 95° C. to denature double stranded nucleic acids, and 22 seconds at 60° C. for primer annealing and extension. Amplicon synthesis was monitored by detecting fluorescence signals in the FAM channel as a function of cycle number.

TABLE 3

Oligonucleotide Probe Reagents

| | | |
|---|---|---|
| Oligo Name | Oligo Function | Oligo Sequence |
| SEQ ID NO:16 | 2058C 16-mer Probe | ACGGCAAGACCCCGTG |
| SEQ ID NO:17 | 2058C 15-mer Probe | ACGGCAAGACCCCGT |
| SEQ ID NO:21 | 2058G 16-mer Probe | ACGGGAAGACCCCGTG |
| SEQ ID NO:22 | 2058G 15-mer Probe | ACGGGAAGACCCCGT |
| SEQ ID NO:26 | 2058T 16-mer Probe | ACGGTAAGACCCCGTG |
| SEQ ID NO:27 | 2058T 15-mer Probe | ACGGTAAGACCCCGT |
| SEQ ID NO:31 | 2059C 16-mer Probe | ACGGACAGACCCCGTG |
| SEQ ID NO:32 | 2059C 15-mer Probe | ACGGACAGACCCCGT |
| SEQ ID NO:36 | 2059G 16-mer Probe | ACGGAGAGACCCCGTG |
| SEQ ID NO:37 | 2059G 15-mer Probe | ACGGAGAGACCCCGT |

The results presented in Table 4 demonstrated that the individual probes behaved differently with respect to assay sensitivity and uniformity of Ct values indicating positivity. The first column in the table identifies the IVT containing a single nucleotide substitution characteristic of macrolide resistance in M. genitalium. Tabulated results include Ct values determined using each of the different probes, and the percentage of positive calls made during the 45 cycle amplification protocol. The initial probe designs presented in Table 1 provided better results in the singleplex reaction format compared to the multiplex procedure of Example 1. The 15-mer probes showed improved sensitivity for the detection of the IVT 2058C, 2059C and 2059G, but did not perform as well as the initial designs (see Table 1) for the detection of IVT 2058G and 2058T. The 16-mer probes showed improvement in sensitivity for detection of all single nucleotide substitutions. Improvement of Ct ranged from 2 to 4 cycles. Moreover, RFU levels for each target nearly tripled by use of the probes presented in Table 3 compared with use of the probes presented in Table 1. Although not shown, specificity of each assay for detecting macrolide resistance was confirmed by the absence of a specific fluorescent signal in the FAM channel when wild-type M. genitalium was used as the source of 23S rRNA templates. Amplification of an internal control template nucleic acid was detected in each reaction mixture, thereby confirming integrity of the amplification and detection procedure.

TABLE 4

Singleplex Real-Time Amplification Results

| | Initial Design (Table 1) | | New Design 15-mers (Table 3) | | New Design 16-mers (Table 3) | |
|---|---|---|---|---|---|---|
| In vitro transcript | 500 c/rxn (Ct: % pos) | 50 c/rxn (Ct: % pos) | 500 c/rxn (Ct: % pos) | 50 c/rxn (Ct: % pos) | 500 c/rxn (Ct: % pos) | 50 c/rxn (Ct: % pos) |
| 2058C | 39.1 : 100 | 43.4 : 100 | 35.8 : 100 | 39.0 : 100 | 35.4 : 100 | 37.4 : 100 |
| 2058G | 40.8 : 100 | 43.4 : 50 | 43.5 : 75 | 44.7 : 25 | 37.0 : 100 | 39.6 : 100 |
| 2058T | 39.3 : 100 | 42.3 : 100 | 41.7 : 100 | 44.6 : 50 | 37.1 : 100 | 40.1 : 100 |
| 2059C | 36.8 : 100 | 39.7 : 100 | 36.0 : 100 | 39.6 : 100 | 34.3 : 100 | 37.9 : 100 |
| 2059G | 39.5 : 100 | 42.9 : 100 | 37.2 : 100 | 41.3 : 100 | 35.5 : 100 | 39.6 : 100 |

Example 3 describes a real-time nucleic acid amplification and detection assay capable of detecting five different single base mutations in the M. genitalium 23S rRNA that are characteristic of macrolide resistance. The markers of drug resistance were all detected using a common (i.e., the same) fluorophore species. The assay did not detect wild-type (macrolide-sensitive) nucleic acids of M. genitalium.

### Example 3

### Multiplex Amplification and Detection of Sequences Characteristic of Macrolide Resistance in M. Genitalium

The Panther Fusion System for automated nucleic acid analysis was again employed for amplifying 23S ribosomal nucleic acid sequences of M. genitalium using the polymerase chain reaction, with monitoring of amplicon production as reaction cycles were occurring. Samples used as sources of amplifiable templates included either 500 copies/reaction or 50 copies/reaction of an IVT corresponding to one of the five macrolide-resistant M. genitalium 23S rRNA sequences. As in the preceding Examples, control reactions used to confirm specificity of the macrolide resistance assays were performed using 23S rRNA templates isolated from 1 × 10⁵ CFU/ml of wild-type (macrolide-sensitive) M. genitalium. An internal control (IC) RNA template, together with primers and a probe for amplifying and detecting the IC also were included. Template nucleic acids were enriched by target capture onto magnetic beads before being combined with enzymes, dNTPs, and cofactors in reaction mixtures that supported reverse transcription and PCR amplification, as will be familiar to those having an ordinary level of skill in the art. Multiplex reaction mixtures (replicates of 6) included forward and reverse primers from Example 1, together with all five labeled oligonucleotide probes. Sequences of oligonucleotide probes used in the procedure are presented in Table 5. Again, probes were labeled with a fluorescent dye at the 5′-end, and with a quencher moiety at the 3′-end, also as described under Example 1. Reaction conditions included: 8 minutes at 46° C. for reverse transcription to synthesize cDNA; a 2 minute 95° C. activation step; 45 cycles of 5 seconds at 95° C. to denature double stranded nucleic acids, and 22 seconds at 60° C. for primer annealing and extension. Amplicon synthesis was monitored by detecting fluorescence signals in the FAM channel as a function of cycle number.

TABLE 5

Oligonucleotide Probe Reagents

| | | |
|---|---|---|
| Oligo Name | Oligo Function | Oligo Sequence |
| SEQ ID NO: 16 | 2058C Probe | ACGGCAAGACCCCGTG |
| SEQ ID NO:21 | 2058G Probe | ACGGGAAGACCCCGTG |
| SEQ ID NO:26 | 2058T Probe | ACGGTAAGACCCCGTG |
| SEQ ID NO:31 | 2059C Probe | ACGGACAGACCCCGTG |
| SEQ ID NO:36 | 2059G Probe | ACGGAGAGACCCCGTG |

Results from the procedure, presented in Table 6, demonstrated that all of the different markers of macrolide resistance in M. genitalium were detectable with substantially equal efficiency. At 500 copies/reaction, all IVT were detected with 100% of positive calls at a Ct between 35.1 and 37.9. At 50 copies/reaction, 4 out of 5 IVTs (IVT 2058C, 2058G, 2059C and 2059G) were detected with 100% of positive calls at a Ct between 37.9 and 40.7. IVT 2058T was detected with 83% positive calls (5 out of 6 replicates) at a mean Ct of 40.8. Although not shown, the same multiplex assay showed no signal in negative specimen transport medium (STM) samples, or in trials conducted using processed samples containing 1 × 10⁵ CFU/ml of wild-type (macrolide sensitive) M. genitalium. STM is a phosphate-buffered detergent solution which, in addition to lysing cells, protects released RNAs by inhibiting the activity of RNases that may be present in the sample undergoing testing. Each CFU (colony forming unit) can be estimated to contain at least 1,000 copies of the target 23S rRNA template. This means that no amplification signal was detectable when the amplification reaction was primed using at least 1 × 10⁸ copies/ml of the target rRNA. Even though no signal was detected for amplification of the M. genitalium target nucleic acid under this condition, a co-amplified internal control was easily detected with an acceptable mean Ct of 33.85, thereby verifying integrity of the amplification and detection procedure.

TABLE 6

Real-Time Amplification Results

| | 500 copies/reaction | | 50 copies/reaction | |
|---|---|---|---|---|
| In vitro transcript | Mean Ct | Pos call % | Mean Ct | Pos call % |
| 2058C | 35.1 | 100 | 37.9 | 100 |
| 2058G | 37.5 | 100 | 40.7 | 100 |
| 2058T | 37.6 | 100 | 40.8 | 83 |
| 2059C | 35.1 | 100 | 38.9 | 100 |
| 2059G | 36.1 | 100 | 38.6 | 100 |

Although various embodiments of the present disclosure have been illustrated and described in detail, it will be readily apparent to those skilled in the art that various modifications may be made without departing from the present disclosure or from the scope of the appended embodiments and claims.

While the present disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the disclosure requires features or combinations of features other than those expressly recited in the embodiments. Accordingly, the present disclosure is deemed to include all modifications and variations encompassed within the spirit and scope of the following numbered embodiments.

### Numbered Embodiments

Embodiment 1 is a method of determining whether a nucleic acid sample isolated from a specimen obtained from a human subject comprises nucleic acids of macrolide-resistant M. genitalium, the method comprising the steps of:
(a) amplifying or having amplified 23S ribosomal nucleic acid sequences that may present in the nucleic acid sample using an in vitro nucleic acid amplification reaction to produce amplicons,
   wherein the in vitro nucleic acid amplification reaction comprises each of
      (i) a DNA polymerase with 5′ to 3′ exonuclease activity,
      (ii) a primer complementary to 23S ribosomal nucleic acids of both macrolide-resistant M. genitalium and macrolide-sensitive M. genitalium, and
      (iii) a collection of two or more oligonucleotide probes,
   wherein the base sequence of at least one oligonucleotide probe among the collection is selected from the group consisting of SEQ ID NO:16, SEQ ID NO:21, SEQ ID NO:26, SEQ ID NO:31, and SEQ ID NO:36,
   wherein each oligonucleotide probe among the collection comprises a fluorophore moiety and a quencher moiety in energy transfer relationship with each other,
   wherein amplicons produced in the in vitro nucleic acid amplification reaction comprise the sequence of any of SEQ ID NO:13, SEQ ID NO:18, SEQ ID NO:23, SEQ ID NO:28, or SEQ ID NO:33 if the nucleic acid sample comprises nucleic acids of macrolide-resistant M. genitalium, and
   wherein amplicons produced in the in vitro nucleic acid amplification reaction comprise the sequence of SEQ ID NO: 11 if the nucleic acid sample comprises nucleic acids of macrolide-sensitive M. genitalium; and
(b) detecting or having detected any of a fluorescent signal produced by the fluorophore moiety of one among the collection of oligonucleotides of the probe reagent in the in vitro nucleic acid amplification reaction,
   whereby if the fluorescent signal is detected then it is determined that the nucleic acid sample comprises nucleic acids of macrolide-resistant M. genitalium, and
   whereby if the fluorescent signal is not detected then it is determined that the nucleic acid sample does not comprise nucleic acids of macrolide-resistant M. genitalium.

Embodiment 2 is the method of embodiment 1, wherein the in vitro nucleic acid amplification reaction comprises a primer extension step carried out at about 60° C.

Embodiment 3 is the method of embodiment 1 or 2, wherein the in vitro nucleic acid amplification reaction of step (a) is a polymerase chain reaction, and wherein step (b) is performed as the polymerase chain reaction is occurring.

Embodiment 4 is the method of any one of embodiments 1 to 3, wherein each of steps (a) and (b) are carried out using an automated nucleic acid analyzer instrument.

Embodiment 5 is the method of any one of embodiments 1 to 4, wherein before step (a) there is a step for preparing the nucleic acid sample, or having the nucleic acid sample prepared, starting with a clinical specimen that may contain M. genitalium cellular material.

Embodiment 6 is the method of embodiment 5, wherein the step for preparing the nucleic acid sample, or having the nucleic acid sample prepared, as well as steps (a) and (b) are carried out using a single automated nucleic acid analyzer instrument.

Embodiment 7 is the method of any one of embodiments 1 to 6, wherein the nucleic acid sample isolated from the specimen obtained from the human subject is known to comprise nucleic acids of M. genitalium before step (a) is conducted.

Embodiment 8 is the method of any one of embodiments 1 to 7, further comprising the step of (c) treating the human subject based on the result of step (b).

Embodiment 9 is the method of embodiment 8,
wherein it is determined in step (b) that the nucleic acid sample comprises nucleic acids of macrolide-resistant M. genitalium, and
wherein step (c) comprises treating the human subject with an antibiotic other than azithromycin.

Embodiment 10 is the method of embodiment 9, wherein the antibiotic other than azithromycin is a fluoroquinolone antibiotic.

Embodiment 11 is the method of any one of embodiments 1 to 6,
wherein the nucleic acid sample isolated from the specimen obtained from the human subject is known to comprise nucleic acids of M. genitalium before step (a) is conducted,
wherein it is determined in step (b) that the nucleic acid sample does not comprise nucleic acids of macrolide-resistant M. genitalium, and
wherein the method further comprises the step of (c) treating the human subject with an antibiotic other than a fluoroquinolone antibiotic.

Embodiment 12 is the method of embodiment 11, wherein the antibiotic other than the fluoroquinolone antibiotic is a macrolide antibiotic.

Embodiment 13 is a probe for detecting nucleic acids of macrolide-resistant M. genitalium but not nucleic acids of macrolide-sensitive M. genitalium, comprising:
an oligonucleotide up to 27 bases in length and comprising 14 contiguous bases of SEQ ID NO:13, including position 11 of SEQ ID NO:13, allowing for substitution of RNA and DNA equivalent bases, and
a detectable label covalently attached to the oligonucleotide.

Embodiment 14 is the probe of embodiment 13, wherein the oligonucleotide is up to 17 bases in length, and wherein the oligonucleotide comprises 14 contiguous bases of SEQ ID NO:14 or the complement thereof, allowing for substitution of RNA and DNA equivalent bases.

Embodiment 15 is the probe of embodiment 13 or 14, wherein the oligonucleotide is up to 17 bases in length, and wherein the oligonucleotide comprises 14 contiguous bases of SEQ ID NO:14 or the complement thereof.

Embodiment 16 is the probe of any one of embodiments 13 to 15, wherein, if included in a template-dependent nucleic acid amplification reaction comprising a primer and a DNA polymerase with 5′ to 3′ exonuclease activity, the oligonucleotide hydrolyzes during extension of the primer when the template being amplified comprises the complement of SEQ ID NO:13, but not when the template being amplified comprises the complement of SEQ ID NO:11.

Embodiment 17 is the probe of embodiment 16, wherein the oligonucleotide hydrolyzes during extension of the primer at 60° C. when the template being amplified comprises the complement of SEQ ID NO:13, but not when the template being amplified comprises the complement of SEQ ID NO: 11.

Embodiment 18 is the probe of any one of embodiments 13 to 17, wherein the detectable label comprises a fluorophore moiety.

Embodiment 19 is the probe of embodiment 18, further comprising a quencher moiety, wherein the quencher moiety is covalently attached to the oligonucleotide, and wherein the fluorophore moiety and the quencher moiety are in energy transfer relationship with each other.

Embodiment 20 is the probe of any one of embodiments 13 to 19, wherein the base sequence of the oligonucleotide is selected from the group consisting of SEQ ID NO:15, SEQ ID NO:16, and SEQ ID NO:17.

Embodiment 21 is the probe of either embodiment 19 or 20, wherein the fluorophore moiety is a fluorescein moiety covalently attached to the 5′-terminal nucleotide of the oligonucleotide, and wherein the quencher moiety is covalently attached to the 3′-terminal nucleotide of the oligonucleotide.

Embodiment 22 is the probe of embodiment 21, wherein the base sequence of the probe is SEQ ID NO:16.

Embodiment 23 is a probe for detecting nucleic acids of macrolide-resistant M. genitalium but not nucleic acids of macrolide-sensitive M. genitalium, comprising:
an oligonucleotide up to 27 bases in length and comprising 15 contiguous bases of SEQ ID NO:18, including position 11 of SEQ ID NO:18, allowing for substitution of RNA and DNA equivalent bases, and
a detectable label covalently attached to the oligonucleotide.

Embodiment 24 is the probe of embodiment 23, wherein the oligonucleotide is up to 18 bases in length, and wherein the oligonucleotide comprises 15 contiguous bases of SEQ ID NO:19 or the complement thereof, allowing for substitution of RNA and DNA equivalent bases.

Embodiment 25 is the probe of embodiment 23 or 24, wherein the oligonucleotide is up to 18 bases in length, and wherein the oligonucleotide comprises 15 contiguous bases of SEQ ID NO:19 or the complement thereof.

Embodiment 26 is the probe of any one of embodiments 23 to 25, wherein, if included in a template-dependent nucleic acid amplification reaction comprising a primer and a DNA polymerase with 5′ to 3′ exonuclease activity, the oligonucleotide hydrolyzes during extension of the primer when the template being amplified comprises the complement of SEQ ID NO:18, but not when the template being amplified comprises the complement of SEQ ID NO: 11.

Embodiment 27 is the probe of embodiment 26, wherein the oligonucleotide hydrolyzes during extension of the primer at 60° C. when the template being amplified comprises the complement of SEQ ID NO:18, but not when the template being amplified comprises the complement of SEQ ID NO: 11.

Embodiment 28 is the probe of any one of embodiments 23 to 27, wherein the detectable label comprises a fluorophore moiety.

Embodiment 29 is the probe of embodiment 28, further comprising a quencher moiety, wherein the quencher moiety is covalently attached to the oligonucleotide, and wherein the fluorophore moiety and the quencher moiety are in energy transfer relationship with each other.

Embodiment 30 is the probe of any one of embodiments 23 to 29, wherein the base sequence of the oligonucleotide is selected from the group consisting of SEQ ID NO:20, SEQ ID NO:21, and SEQ ID NO:22.

Embodiment 31 is the probe of either embodiment 29 or 30, wherein the fluorophore moiety is a fluorescein moiety covalently attached to the 5′-terminal nucleotide of the oligonucleotide, and wherein the quencher moiety is covalently attached to the 3′-terminal nucleotide of the oligonucleotide.

Embodiment 32 is the probe of embodiment 31, wherein the base sequence of the probe is SEQ ID NO:21.

Embodiment 33 is a probe for detecting nucleic acids of macrolide-resistant M. genitalium but not nucleic acids of macrolide-sensitive M. genitalium, comprising:
an oligonucleotide up to 27 bases in length and comprising 15 contiguous bases of SEQ ID NO:23, including position 11 of SEQ ID NO:23, allowing for substitution of RNA and DNA equivalent bases, and
a detectable label covalently attached to the oligonucleotide.

Embodiment 34 is the probe of embodiment 33, wherein the oligonucleotide is up to 19 bases in length, and wherein the oligonucleotide comprises 15 contiguous bases of SEQ ID NO:24 or the complement thereof, allowing for substitution of RNA and DNA equivalent bases.

Embodiment 35 is the probe of embodiment 33 or 34, wherein the oligonucleotide is up to 19 bases in length, and wherein the oligonucleotide comprises 15 contiguous bases of SEQ ID NO:24 or the complement thereof.

Embodiment 36 is the probe of any one of embodiments 33 to 35, wherein, if included in a template-dependent nucleic acid amplification reaction comprising a primer and a DNA polymerase with 5′ to 3′ exonuclease activity, the oligonucleotide hydrolyzes during extension of the primer when the template being amplified comprises the complement of SEQ ID NO:23, but not when the template being amplified comprises the complement of SEQ ID NO: 11.

Embodiment 37 is the probe of embodiment 36, wherein the oligonucleotide hydrolyzes during extension of the primer at 60° C. when the template being amplified comprises the complement of SEQ ID NO:23, but not when the template being amplified comprises the complement of SEQ ID NO: 11.

Embodiment 38 is the probe of any one of embodiments 33 to 37, wherein the detectable label comprises a fluorophore moiety.

Embodiment 39 is the probe of embodiment 38, further comprising a quencher moiety, wherein the quencher moiety is covalently attached to the oligonucleotide, and wherein the fluorophore moiety and the quencher moiety are in energy transfer relationship with each other.

Embodiment 40 is the probe of any one of embodiments 33 to 39, wherein the base sequence of the oligonucleotide is selected from the group consisting of SEQ ID NO:25, SEQ ID NO:26, and SEQ ID NO:27.

Embodiment 41 is the probe of either embodiment 39 or 40, wherein the fluorophore moiety is a fluorescein moiety covalently attached to the 5′-terminal nucleotide of the oligonucleotide, and wherein the quencher moiety is covalently attached to the 3′-terminal nucleotide of the oligonucleotide.

Embodiment 42 is the probe of embodiment 41, wherein the base sequence of the probe is SEQ ID NO:26.

Embodiment 43 is a probe for detecting nucleic acids of macrolide-resistant M. genitalium but not nucleic acids of macrolide-sensitive M. genitalium, comprising:
an oligonucleotide up to 27 bases in length and comprising 15 contiguous bases of SEQ ID NO:28, including position 12 of SEQ ID NO:28, allowing for substitution of RNA and DNA equivalent bases, and
a detectable label covalently attached to the oligonucleotide.

Embodiment 44 is the probe of embodiment 43, wherein the oligonucleotide is up to 19 bases in length, and wherein the oligonucleotide comprises 15 contiguous bases of SEQ ID NO:29 or the complement thereof, allowing for substitution of RNA and DNA equivalent bases.

Embodiment 45 is the probe of either embodiment 43 or embodiment 44, wherein the oligonucleotide is up to 19 bases in length, and wherein the oligonucleotide comprises 15 contiguous bases of SEQ ID NO:29 or the complement thereof.

Embodiment 46 is the probe of any one of embodiments 43 to 45, wherein, if included in a template-dependent nucleic acid amplification reaction comprising a primer and a DNA polymerase with 5′ to 3′ exonuclease activity, the oligonucleotide hydrolyzes during extension of the primer when the template being amplified comprises the complement of SEQ ID NO:28, but not when the template being amplified comprises the complement of SEQ ID NO: 11.

Embodiment 47 is the probe of embodiment 46, wherein the oligonucleotide hydrolyzes during extension of the primer at 60° C. when the template being amplified comprises the complement of SEQ ID NO:28, but not when the template being amplified comprises the complement of SEQ ID NO: 11.

Embodiment 48 is the probe of any one of embodiments 43 to 47, wherein the detectable label comprises a fluorophore moiety.

Embodiment 49 is the probe of embodiment 48, further comprising a quencher moiety, wherein the quencher moiety is covalently attached to the oligonucleotide, and wherein the fluorophore moiety and the quencher moiety are in energy transfer relationship with each other.

Embodiment 50 is the probe of any one of embodiments 43 to 49, wherein the base sequence of the oligonucleotide is selected from the group consisting of SEQ ID NO:30, SEQ ID NO:31, and SEQ ID NO:32.

Embodiment 51 is the probe of either embodiment 49 or 50, wherein the fluorophore moiety is a fluorescein moiety covalently attached to the 5′-terminal nucleotide of the oligonucleotide, and wherein the quencher moiety is covalently attached to the 3′-terminal nucleotide of the oligonucleotide.

Embodiment 52 is the probe of embodiment 51, wherein the base sequence of the probe is SEQ ID NO:31.

Embodiment 53 is a probe for detecting nucleic acids of macrolide-resistant M. genitalium but not nucleic acids of macrolide-sensitive M. genitalium, comprising:
an oligonucleotide up to 27 bases in length and comprising 15 contiguous bases of SEQ ID NO:33, including position 12 of SEQ ID NO:33, allowing for substitution of RNA and DNA equivalent bases, and
a detectable label covalently attached to the oligonucleotide.

Embodiment 54 is the probe of embodiment 53, wherein the oligonucleotide is up to 18 bases in length, and wherein the oligonucleotide comprises 15 contiguous bases of SEQ ID NO:34 or the complement thereof, allowing for substitution of RNA and DNA equivalent bases.

Embodiment 55 is the probe of embodiment 53 or 54 wherein the oligonucleotide is up to 18 bases in length, and wherein the oligonucleotide comprises 15 contiguous bases of SEQ ID NO:34 or the complement thereof.

Embodiment 56 is the probe of any one of embodiments 53 to 55, wherein, if included in a template-dependent nucleic acid amplification reaction comprising a primer and a DNA polymerase with 5′ to 3′ exonuclease activity, the oligonucleotide hydrolyzes during extension of the primer when the template being amplified comprises the complement of SEQ ID NO:33, but not when the template being amplified comprises the complement of SEQ ID NO: 11.

Embodiment 57 is the probe of embodiment 56, wherein the oligonucleotide hydrolyzes during extension of the primer at 60° C. when the template being amplified comprises the complement of SEQ ID NO:33, but not when the template being amplified comprises the complement of SEQ ID NO: 11.

Embodiment 58 is the probe of any one of embodiments 53 to 57, wherein the detectable label comprises a fluorophore moiety.

Embodiment 59 is the probe of embodiment 58, further comprising a quencher moiety, wherein the quencher moiety is covalently attached to the oligonucleotide, and wherein the fluorophore moiety and the quencher moiety are in energy transfer relationship with each other.

Embodiment 60 is the probe of any one of embodiments 53 to 59, wherein the base sequence of the oligonucleotide is selected from the group consisting of SEQ ID NO:35, SEQ ID NO:36, and SEQ ID NO:37.

Embodiment 61 is the probe of either embodiment 59 of 60, wherein the fluorophore moiety is a fluorescein moiety covalently attached to the 5′-terminal nucleotide of the oligonucleotide, and wherein the quencher moiety is covalently attached to the 3′-terminal nucleotide of the oligonucleotide.

Embodiment 62 is the probe of embodiment 61, wherein the base sequence of the probe is SEQ ID NO:36.

Embodiment 63 is a probe reagent for detecting nucleic acids of macrolide-resistant M. genitalium, comprising:
a collection of two or more oligonucleotide probes,
   wherein the base sequence of at least one oligonucleotide probe among the collection is selected from the group consisting of SEQ ID NO:16, SEQ ID NO:21, SEQ ID NO:26, SEQ ID NO:31, and SEQ ID NO:36, and
   wherein each oligonucleotide probe among the collection comprises a fluorophore moiety and a quencher moiety in energy transfer relationship with each other.

Embodiment 64 is the probe reagent of embodiment 63, wherein the base sequences of at least two oligonucleotide probes of the collection are selected from the group consisting of SEQ ID NO:16, SEQ ID NO:21, SEQ ID NO:26, SEQ ID NO:31, and SEQ ID NO:36.

Embodiment 65 is the probe reagent of either embodiment 63 or embodiment 64, wherein, if the collection of oligonucleotide probes is included in a template-dependent nucleic acid amplification reaction comprising a primer and a DNA polymerase with 5′ to 3′ exonuclease activity, an oligonucleotide probe from among the collection hydrolyzes during extension of the primer when the template being amplified comprises the complement of any of SEQ ID NO:13, SEQ ID NO:18, SEQ ID NO:23, SEQ ID NO:28, or SEQ ID NO:33, but not when the template being amplified comprises the complement of SEQ ID NO: 11.

Embodiment 66 is the probe reagent of embodiment 65, wherein the oligonucleotide probe from among the collection hydrolyzes during extension of the primer at about 60° C.

Embodiment 67 is the probe reagent of any one of embodiments 63 to 66, wherein the fluorophore moiety of each different oligonucleotide probe is attached to a terminal nucleotide thereof.

Embodiment 68 is the probe reagent of any one of embodiments 63 to 67, wherein the fluorophore moiety is a fluorescein moiety.

Embodiment 69 is the probe reagent of any one of embodiments 63 to 68, wherein the quencher moiety is the same for each of the oligonucleotide probes among the collection of two or more oligonucleotide probes.

The invention has been described with reference to a number of specific examples and embodiments. Of course, a number of different embodiments of the present invention will suggest themselves to those having ordinary skill in the art upon review of the foregoing description. Thus, the true scope of the present invention is to be determined upon reference to the appended claims.

What is claimed is:

1. A method of determining whether a nucleic acid sample isolated from a specimen obtained from a human subject comprises nucleic acids of macrolide-resistant M. genitalium, the method comprising the steps of:
(a) amplifying or having amplified 23S ribosomal nucleic acid sequences that may present in the nucleic acid sample using an in vitro nucleic acid amplification reaction to produce amplicons,
wherein the in vitro nucleic acid amplification reaction comprises each of
(i) a DNA polymerase with 5′ to 3′ exonuclease activity,
(ii) a primer complementary to 23S ribosomal nucleic acids of both macrolide-resistant M. genitalium and macrolide-sensitive M. genitalium, and
(iii) a collection of two or more oligonucleotide probes, wherein the base sequence of at least one oligonucleotide probe among the collection is selected from the group consisting of SEQ ID NO:16, SEQ ID NO:21, SEQ ID NO:26, SEQ ID NO:31, and SEQ ID NO:36,
wherein each oligonucleotide probe among the collection comprises a fluorophore moiety and a quencher moiety in energy transfer relationship with each other,
wherein amplicons produced in the in vitro nucleic acid amplification reaction comprise the sequence of any of SEQ ID NO:13, SEQ ID NO:18, SEQ ID NO:23, SEQ ID NO:28, or SEQ ID NO:33 if the nucleic acid sample comprises nucleic acids of macrolide-resistant M. genitalium, and
wherein amplicons produced in the in vitro nucleic acid amplification reaction comprise the sequence of SEQ ID NO: 11 if the nucleic acid sample comprises nucleic acids of macrolide-sensitive M. genitalium; and
(b) detecting or having detected any of a fluorescent signal produced by the fluorophore moiety of one among the collection of oligonucleotides of the probe reagent in the in vitro nucleic acid amplification reaction,
whereby if the fluorescent signal is detected then it is determined that the nucleic acid sample comprises nucleic acids of macrolide-resistant M. genitalium, and
whereby if the fluorescent signal is not detected then it is determined that the nucleic acid sample does not comprise nucleic acids of macrolide-resistant M. genitalium.

2. The method of claim 1, wherein the in vitro nucleic acid amplification reaction comprises a primer extension step carried out at about 60° C.

3. The method of claim 1 or 2, wherein the in vitro nucleic acid amplification reaction of step (a) is a polymerase chain reaction, and wherein step (b) is performed as the polymerase chain reaction is occurring.

4. The method of any one of claims 1 to 3, wherein each of steps (a) and (b) are carried out using an automated nucleic acid analyzer instrument.

5. The method of any one of claims 1 to 4, wherein before step (a) there is a step for preparing the nucleic acid sample, or having the nucleic acid sample prepared, starting with a clinical specimen that may contain M. genitalium cellular material.

6. The method of claim 5, wherein the step for preparing the nucleic acid sample, or having the nucleic acid sample prepared, as well as steps (a) and (b) are carried out using a single automated nucleic acid analyzer instrument.

7. The method of any one of claims 1 to 6, wherein the nucleic acid sample isolated from the specimen obtained from the human subject is known to comprise nucleic acids of M. genitalium before step (a) is conducted.

8. The method of any one of claims 1 to 7, further comprising the step of (c) treating the human subject based on the result of step (b).

9. The method of claim 8,
wherein it is determined in step (b) that the nucleic acid sample comprises nucleic acids of macrolide-resistant M. genitalium, and
wherein step (c) comprises treating the human subject with an antibiotic other than azithromycin.

10. The method of claim 9, wherein the antibiotic other than azithromycin is a fluoroquinolone antibiotic.

11. The method of any one of claims 1 to 6,
wherein the nucleic acid sample isolated from the specimen obtained from the human subject is known to comprise nucleic acids of M. genitalium before step (a) is conducted,
wherein it is determined in step (b) that the nucleic acid sample does not comprise nucleic acids of macrolide-resistant M. genitalium, and
wherein the method further comprises the step of (c) treating the human subject with an antibiotic other than a fluoroquinolone antibiotic.

12. The method of claim 11, wherein the antibiotic other than the fluoroquinolone antibiotic is a macrolide antibiotic.

13. A probe for detecting nucleic acids of macrolide-resistant M. genitalium but not nucleic acids of macrolide-sensitive M. genitalium, comprising:
an oligonucleotide up to 27 bases in length and comprising 14 contiguous bases of SEQ ID NO:13, including position 11 of SEQ ID NO:13, allowing for substitution of RNA and DNA equivalent bases, and
a detectable label covalently attached to the oligonucleotide.

14. The probe of claim 13, wherein the oligonucleotide is up to 17 bases in length, and wherein the oligonucleotide comprises 14 contiguous bases of SEQ ID NO:14 or the complement thereof, allowing for substitution of RNA and DNA equivalent bases.

15. The probe of claim 13 or 14, wherein the oligonucleotide is up to 17 bases in length, and wherein the oligonucleotide comprises 14 contiguous bases of SEQ ID NO:14 or the complement thereof.

16. The probe of any one of claims 13 to 15, wherein, if included in a template-dependent nucleic acid amplification reaction comprising a primer and a DNA polymerase with 5′ to 3′ exonuclease activity, the oligonucleotide hydrolyzes during extension of the primer when the template being amplified comprises the complement of SEQ ID NO:13, but not when the template being amplified comprises the complement of SEQ ID NO:11.

17. The probe of claim 16, wherein the oligonucleotide hydrolyzes during extension of the primer at 60° C. when the template being amplified comprises the complement of SEQ ID NO:13, but not when the template being amplified comprises the complement of SEQ ID NO:11.

18. The probe of any one of claims 13 to 17, wherein the detectable label comprises a fluorophore moiety.

19. The probe of claim 18, further comprising a quencher moiety, wherein the quencher moiety is covalently attached to the oligonucleotide, and wherein the fluorophore moiety and the quencher moiety are in energy transfer relationship with each other.

20. The probe of any one of claims 13 to 19, wherein the base sequence of the oligonucleotide is selected from the group consisting of SEQ ID NO:15, SEQ ID NO:16, and SEQ ID NO:17.

21. The probe of either claim 19 or 20, wherein the fluorophore moiety is a fluorescein moiety covalently attached to the 5′-terminal nucleotide of the oligonucleotide, and wherein the quencher moiety is covalently attached to the 3′-terminal nucleotide of the oligonucleotide.

22. The probe of claim 21, wherein the base sequence of the probe is SEQ ID NO:16.

23. A probe for detecting nucleic acids of macrolide-resistant M. genitalium but not nucleic acids of macrolide-sensitive M. genitalium, comprising:
an oligonucleotide up to 27 bases in length and comprising 15 contiguous bases of SEQ ID NO:18, including position 11 of SEQ ID NO:18, allowing for substitution of RNA and DNA equivalent bases, and
a detectable label covalently attached to the oligonucleotide.

24. The probe of claim 23, wherein the oligonucleotide is up to 18 bases in length, and wherein the oligonucleotide comprises 15 contiguous bases of SEQ ID NO:19 or the complement thereof, allowing for substitution of RNA and DNA equivalent bases.

25. The probe of claim 23 or 24, wherein the oligonucleotide is up to 18 bases in length, and wherein the oligonucleotide comprises 15 contiguous bases of SEQ ID NO:19 or the complement thereof.

26. The probe of any one of claims 23 to 25, wherein, if included in a template-dependent nucleic acid amplification reaction comprising a primer and a DNA polymerase with 5′ to 3′ exonuclease activity, the oligonucleotide hydrolyzes during extension of the primer when the template being amplified comprises the complement of SEQ ID NO:18, but not when the template being amplified comprises the complement of SEQ ID NO:11.

27. The probe of claim 26, wherein the oligonucleotide hydrolyzes during extension of the primer at 60° C. when the template being amplified comprises the complement of SEQ ID NO:18, but not when the template being amplified comprises the complement of SEQ ID NO:11.

28. The probe of any one of claims 23 to 27, wherein the detectable label comprises a fluorophore moiety.

29. The probe of claim 28, further comprising a quencher moiety, wherein the quencher moiety is covalently attached to the oligonucleotide, and wherein the fluorophore moiety and the quencher moiety are in energy transfer relationship with each other.

30. The probe of any one of claims 23 to 29, wherein the base sequence of the oligonucleotide is selected from the group consisting of SEQ ID NO:20, SEQ ID NO:21, and SEQ ID NO:22.

31. The probe of either claim 29 or 30, wherein the fluorophore moiety is a fluorescein moiety covalently attached to the 5′-terminal nucleotide of the oligonucleotide, and wherein the quencher moiety is covalently attached to the 3′-terminal nucleotide of the oligonucleotide.

32. The probe of claim 31, wherein the base sequence of the probe is SEQ ID NO:21.

33. A probe for detecting nucleic acids of macrolide-resistant M. genitalium but not nucleic acids of macrolide-sensitive M. genitalium, comprising:
an oligonucleotide up to 27 bases in length and comprising 15 contiguous bases of SEQ ID NO:23, including position 11 of SEQ ID NO:23, allowing for substitution of RNA and DNA equivalent bases, and
a detectable label covalently attached to the oligonucleotide.

34. The probe of claim 33, wherein the oligonucleotide is up to 19 bases in length, and wherein the oligonucleotide comprises 15 contiguous bases of SEQ ID NO:24 or the complement thereof, allowing for substitution of RNA and DNA equivalent bases.

35. The probe of claim 33 or 34, wherein the oligonucleotide is up to 19 bases in length, and wherein the oligonucleotide comprises 15 contiguous bases of SEQ ID NO:24 or the complement thereof.

36. The probe of any one of claims 33 to 35, wherein, if included in a template-dependent nucleic acid amplification reaction comprising a primer and a DNA polymerase with 5′ to 3′ exonuclease activity, the oligonucleotide hydrolyzes during extension of the primer when the template being amplified comprises the complement of SEQ ID NO:23, but not when the template being amplified comprises the complement of SEQ ID NO:11.

37. The probe of claim 36, wherein the oligonucleotide hydrolyzes during extension of the primer at 60° C. when the template being amplified comprises the complement of SEQ ID NO:23, but not when the template being amplified comprises the complement of SEQ ID NO:11.

38. The probe of any one of claims 33 to 37, wherein the detectable label comprises a fluorophore moiety.

39. The probe of claim 38, further comprising a quencher moiety, wherein the quencher moiety is covalently attached to the oligonucleotide, and wherein the fluorophore moiety and the quencher moiety are in energy transfer relationship with each other.

40. The probe of any one of claims 33 to 39, wherein the base sequence of the oligonucleotide is selected from the group consisting of SEQ ID NO:25, SEQ ID NO:26, and SEQ ID NO:27.

41. The probe of either claim 39 or 40, wherein the fluorophore moiety is a fluorescein moiety covalently attached to the 5′-terminal nucleotide of the oligonucleotide, and wherein the quencher moiety is covalently attached to the 3′-terminal nucleotide of the oligonucleotide.

42. The probe of claim 41, wherein the base sequence of the probe is SEQ ID NO:26.

43. A probe for detecting nucleic acids of macrolide-resistant M. genitalium but not nucleic acids of macrolide-sensitive M. genitalium, comprising:
an oligonucleotide up to 27 bases in length and comprising 15 contiguous bases of SEQ ID NO:28, including position 12 of SEQ ID NO:28, allowing for substitution of RNA and DNA equivalent bases, and
a detectable label covalently attached to the oligonucleotide.

44. The probe of claim 43, wherein the oligonucleotide is up to 19 bases in length, and wherein the oligonucleotide comprises 15 contiguous bases of SEQ ID NO:29 or the complement thereof, allowing for substitution of RNA and DNA equivalent bases.

45. The probe of either claim 43 or claim 44, wherein the oligonucleotide is up to 19 bases in length, and wherein the oligonucleotide comprises 15 contiguous bases of SEQ ID NO:29 or the complement thereof.

46. The probe of any one of claims 43 to 45, wherein, if included in a template-dependent nucleic acid amplification reaction comprising a primer and a DNA polymerase with 5′ to 3′ exonuclease activity, the oligonucleotide hydrolyzes during extension of the primer when the template being amplified comprises the complement of SEQ ID NO:28, but not when the template being amplified comprises the complement of SEQ ID NO:11.

47. The probe of claim 46, wherein the oligonucleotide hydrolyzes during extension of the primer at 60° C. when the template being amplified comprises the complement of SEQ ID NO:28, but not when the template being amplified comprises the complement of SEQ ID NO:11.

48. The probe of any one of claims 43 to 47, wherein the detectable label comprises a fluorophore moiety.

49. The probe of claim 48, further comprising a quencher moiety, wherein the quencher moiety is covalently attached to the oligonucleotide, and wherein the fluorophore moiety and the quencher moiety are in energy transfer relationship with each other.

50. The probe of any one of claims 43 to 49, wherein the base sequence of the oligonucleotide is selected from the group consisting of SEQ ID NO:30, SEQ ID NO:31, and SEQ ID NO:32.

51. The probe of either claim 49 or 50, wherein the fluorophore moiety is a fluorescein moiety covalently attached to the 5′-terminal nucleotide of the oligonucleotide, and wherein the quencher moiety is covalently attached to the 3′-terminal nucleotide of the oligonucleotide.

52. The probe of claim 51, wherein the base sequence of the probe is SEQ ID NO:31.

53. A probe for detecting nucleic acids of macrolide-resistant M. genitalium but not nucleic acids of macrolide-sensitive M. genitalium, comprising:
an oligonucleotide up to 27 bases in length and comprising 15 contiguous bases of SEQ ID NO:33, including position 12 of SEQ ID NO:33, allowing for substitution of RNA and DNA equivalent bases, and
a detectable label covalently attached to the oligonucleotide.

54. The probe of claim 53, wherein the oligonucleotide is up to 18 bases in length, and wherein the oligonucleotide comprises 15 contiguous bases of SEQ ID NO:34 or the complement thereof, allowing for substitution of RNA and DNA equivalent bases.

55. The probe of claim 53 or 54 wherein the oligonucleotide is up to 18 bases in length, and wherein the oligonucleotide comprises 15 contiguous bases of SEQ ID NO:34 or the complement thereof.

56. The probe of any one of claims 53 to 55, wherein, if included in a template-dependent nucleic acid amplification reaction comprising a primer and a DNA polymerase with 5′ to 3′ exonuclease activity, the oligonucleotide hydrolyzes during extension of the primer when the template being amplified comprises the complement of SEQ ID NO:33, but not when the template being amplified comprises the complement of SEQ ID NO:11.

57. The probe of claim 56, wherein the oligonucleotide hydrolyzes during extension of the primer at 60° C. when the template being amplified comprises the complement of SEQ ID NO:33, but not when the template being amplified comprises the complement of SEQ ID NO:11.

58. The probe of any one of claims 53 to 57, wherein the detectable label comprises a fluorophore moiety.

59. The probe of claim 58, further comprising a quencher moiety, wherein the quencher moiety is covalently attached to the oligonucleotide, and wherein the fluorophore moiety and the quencher moiety are in energy transfer relationship with each other.

60. The probe of any one of claims 53 to 59, wherein the base sequence of the oligonucleotide is selected from the group consisting of SEQ ID NO:35, SEQ ID NO:36, and SEQ ID NO:37.

61. The probe of either claim 59 or 60, wherein the fluorophore moiety is a fluorescein moiety covalently attached to the 5′-terminal nucleotide of the oligonucleotide, and wherein the quencher moiety is covalently attached to the 3′-terminal nucleotide of the oligonucleotide.

62. The probe of claim 61, wherein the base sequence of the probe is SEQ ID NO:36.

63. A probe reagent for detecting nucleic acids of macrolide-resistant M. genitalium, comprising:
a collection of two or more oligonucleotide probes,
wherein the base sequence of at least one oligonucleotide probe among the collection is selected from the group consisting of SEQ ID NO:16, SEQ ID NO:21, SEQ ID NO:26, SEQ ID NO:31, and SEQ ID NO:36, and
wherein each oligonucleotide probe among the collection comprises a fluorophore moiety and a quencher moiety in energy transfer relationship with each other.

64. The probe reagent of claim 63, wherein the base sequences of at least two oligonucleotide probes of the collection are selected from the group consisting of SEQ ID NO:16, SEQ ID NO:21, SEQ ID NO:26, SEQ ID NO:31, and SEQ ID NO:36.

65. The probe reagent of either claim 63 or claim 64, wherein, if the collection of oligonucleotide probes is included in a template-dependent nucleic acid amplification reaction comprising a primer and a DNA polymerase with 5′ to 3′ exonuclease activity, an oligonucleotide probe from among the collection hydrolyzes during extension of the primer when the template being amplified comprises the complement of any of SEQ ID NO:13, SEQ ID NO:18, SEQ ID NO:23, SEQ ID NO:28, or SEQ ID NO:33, but not when the template being amplified comprises the complement of SEQ ID NO:11.

66. The probe reagent of claim 65, wherein the oligonucleotide probe from among the collection hydrolyzes during extension of the primer at about 60° C.

67. The probe reagent of any one of claims 63 to 66, wherein the fluorophore moiety of each different oligonucleotide probe is attached to a terminal nucleotide thereof.

68. The probe reagent of any one of claims 63 to 67, wherein the fluorophore moiety is a fluorescein moiety.

69. The probe reagent of any one of claims 63 to 68, wherein the quencher moiety is the same for each of the oligonucleotide probes among the collection of two or more oligonucleotide probes.


SEQUENCE LISTING (37 sequences)

SEQ ID NO: 1 - DNA, 210 bp, Mycoplasma genitalium
  misc_feature: Wildtype M. genitalium sequence
ttgactgtctcggctatagactcggtgaaatccaggtacgggtgaagacacccgttaggcgcaacgggacggaaagaccccgtgaagctttactgtagcttaatattgatcaaaacaccaccatgtagagaataggtaggagcaattgatgcaagttcgcaaggatttgttgatgtgaaatgtggaatactacccttggttatgttttgt

SEQ ID NO: 2 - DNA, 210 bp, Mycoplasma genitalium
  misc_feature: 2058C template
ttgactgtctcggctatagactcggtgaaatccaggtacgggtgaagacacccgttaggcgcaacgggacggcaagaccccgtgaagctttactgtagcttaatattgatcaaaacaccaccatgtagagaataggtaggagcaattgatgcaagttcgcaaggatttgttgatgtgaaatgtggaatactacccttggttatgttttgt

SEQ ID NO: 3 - DNA, 210 bp, Mycoplasma genitalium
  misc_feature: 2058G template
ttgactgtctcggctatagactcggtgaaatccaggtacgggtgaagacacccgttaggcgcaacgggacgggaagaccccgtgaagctttactgtagcttaatattgatcaaaacaccaccatgtagagaataggtaggagcaattgatgcaagttcgcaaggatttgttgatgtgaaatgtggaatactacccttggttatgttttgt

SEQ ID NO: 4 - DNA, 210 bp, Mycoplasma genitalium
  misc_feature: 2058T template
ttgactgtctcggctatagactcggtgaaatccaggtacgggtgaagacacccgttaggcgcaacgggacggtaagaccccgtgaagctttactgtagcttaatattgatcaaaacaccaccatgtagagaataggtaggagcaattgatgcaagttcgcaaggatttgttgatgtgaaatgtggaatactacccttggttatgttttgt

SEQ ID NO: 5 - DNA, 210 bp, Mycoplasma genitalium
  misc_feature: 2059C template
ttgactgtctcggctatagactcggtgaaatccaggtacgggtgaagacacccgttaggcgcaacgggacggacagaccccgtgaagctttactgtagcttaatattgatcaaaacaccaccatgtagagaataggtaggagcaattgatgcaagttcgcaaggatttgttgatgtgaaatgtggaatactacccttggttatgttttgt

SEQ ID NO: 6 - DNA, 210 bp, Mycoplasma genitalium
  misc_feature: 2059G template
ttgactgtctcggctatagactcggtgaaatccaggtacgggtgaagacacccgttaggcgcaacgggacggagagaccccgtgaagctttactgtagcttaatattgatcaaaacaccaccatgtagagaataggtaggagcaattgatgcaagttcgcaaggatttgttgatgtgaaatgtggaatactacccttggttatgttttgt

SEQ ID NO: 7 - DNA, 30 bp, Mycoplasma genitalium
  misc_feature: Forward primer domain
atagactcggtgaaatccaggtacgggtga

SEQ ID NO: 8 - DNA, 20 bp, Mycoplasma genitalium
  misc_feature: Forward primer
ctcggtgaaatccaggtacg

SEQ ID NO: 9 - DNA, 35 bp, Mycoplasma genitalium
  misc_feature: Reverse primer domain
gggtagtattccacatttcacatcaacaaatcctt

SEQ ID NO: 10 - DNA, 25 bp, Mycoplasma genitalium
  misc_feature: Reverse primer
gtattccacatttcacatcaacaaa

SEQ ID NO: 11 - DNA, 27 bp, Mycoplasma genitalium
  misc_feature: Segment of wild-type amplicon
aacgggacggaaagaccccgtgaagct

SEQ ID NO: 12 - DNA, 27 bp, Mycoplasma genitalium
  misc_feature: Segment of amplicon encoding macrolide resistance
  misc_feature (11)..(11): Position 11 is any of C, G, or T
  misc_feature (12)..(12): Position 12 is either C or G
aacgggacggnnagaccccgtgaagct

SEQ ID NO: 13 - DNA, 27 bp, Mycoplasma genitalium
  misc_feature: 2058C probe domain
aacgggacggcaagaccccgtgaagct

SEQ ID NO: 14 - DNA, 17 bp, Mycoplasma genitalium
  misc_feature: 2058C probe subdomain
gacggcaagaccccgtg

SEQ ID NO: 15 - DNA, 14 bp, Mycoplasma genitalium
  misc_feature: 2058C probe
gacggcaagacccc

SEQ ID NO: 16 - DNA, 16 bp, Mycoplasma genitalium
  misc_feature: 2058C probe
acggcaagaccccgtg

SEQ ID NO: 17 - DNA, 15 bp, Mycoplasma genitalium
  misc_feature: 2058C probe
acggcaagaccccgt

SEQ ID NO: 18 - DNA, 27 bp, Mycoplasma genitalium
  misc_feature: 2058G probe domain
aacgggacgggaagaccccgtgaagct

SEQ ID NO: 19 - DNA, 18 bp, Mycoplasma genitalium
  misc_feature: 2058G probe subdomain
ggacgggaagaccccgtg

SEQ ID NO: 20 - DNA, 15 bp, Mycoplasma genitalium
  misc_feature: 2058G probe
ggacgggaagacccc

SEQ ID NO: 21 - DNA, 16 bp, Mycoplasma genitalium
  misc_feature: 2058G probe
acgggaagaccccgtg

SEQ ID NO: 22 - DNA, 15 bp, Mycoplasma genitalium
  misc_feature: 2058G probe
acgggaagaccccgt

SEQ ID NO: 23 - DNA, 27 bp, Mycoplasma genitalium
  misc_feature: 2058T probe domain
aacgggacggtaagaccccgtgaagct

SEQ ID NO: 24 - DNA, 19 bp, Mycoplasma genitalium
  misc_feature: 2058T probe subdomain
gggacggtaagaccccgtg

SEQ ID NO: 25 - DNA, 16 bp, Mycoplasma genitalium
  misc_feature: 2058T probe
gggacggtaagacccc

SEQ ID NO: 26 - DNA, 16 bp, Mycoplasma genitalium
  misc_feature: 2058T probe
acggtaagaccccgtg

SEQ ID NO: 27 - DNA, 15 bp, Mycoplasma genitalium
  misc_feature: 2058T probe
acggtaagaccccgt

SEQ ID NO: 28 - DNA, 27 bp, Mycoplasma genitalium
  misc_feature: 2059C probe domain
aacgggacggacagaccccgtgaagct

SEQ ID NO: 29 - DNA, 19 bp, Mycoplasma genitalium
  misc_feature: 2059C probe subdomain
gggacggacagaccccgtg

SEQ ID NO: 30 - DNA, 16 bp, Mycoplasma genitalium
  misc_feature: 2059C probe
gggacggacagacccc

SEQ ID NO: 31 - DNA, 16 bp, Mycoplasma genitalium
  misc_feature: 2059C probe
acggacagaccccgtg

SEQ ID NO: 32 - DNA, 15 bp, Mycoplasma genitalium
  misc_feature: 2059C probe
acggacagaccccgt

SEQ ID NO: 33 - DNA, 27 bp, Mycoplasma genitalium
  misc_feature: 2059G probe domain
aacgggacggagagaccccgtgaagct

SEQ ID NO: 34 - DNA, 18 bp, Mycoplasma genitalium
  misc_feature: 2059G probe subdomain
ggacggagagaccccgtg

SEQ ID NO: 35 - DNA, 15 bp, Mycoplasma genitalium
  misc_feature: 2059G probe
ggacggagagacccc

SEQ ID NO: 36 - DNA, 16 bp, Mycoplasma genitalium
  misc_feature: 2059G probe
acggagagaccccgtg

SEQ ID NO: 37 - DNA, 15 bp, Mycoplasma genitalium
  misc_feature: 2059G probe
acggagagaccccgt